(19) United States
(12) Patent Application Publication
(10) Pub. No.: US 2011/0257031 A1
(43) Pub. Date: Oct. 20, 2011

(54) Nucleic acid, biomolecule and polymer identifier codes

(75) Inventors: John BODEAU, San Mateo, CA (US); Heinz BREU, Palo Alto, CA (US); Kathleen PERRY, San Francisco, CA (US); Adam HARRIS, Carlsbad, CA (US); Patrick GILLES, Carlsbad, CA (US); Miho Gilles, Carsbad, CA (US)

(73) Assignee: LIFE TECHNOLOGIES CORPORATION, Carlsbad, CA (US)

(21) Appl. No.: 13/026,046

(22) Filed: Feb. 11, 2011

Related U.S. Application Data
(60) Provisional application No. 61/303,954, filed on Feb. 12, 2010.
(60) Provisional application No. 61/307,348, filed on Feb. 23, 2010.
(60) Provisional application No. 61/314,554, filed on Mar. 16, 2010.
(60) Provisional application No. 61/356,491, filed on Jun. 18, 2010.
(60) Provisional application No. 61/391,574, filed on Oct. 8, 2010.

(51) Int. Cl. C40B 30/04; C07H 21/04; C40B 40/06

(52) U.S. Cl. 506/9; 506/16; 536/24.31

(57) ABSTRACT

Provided herein are systems, compositions and methods for tracking, sorting and/or identifying sample polynucleotides using nucleic acid barcodes. The barcodes provided herein are oligonucleotides that are designed to be uniquely identifiable. The nucleic acid barcodes have properties that permit them to be sequenced with high accuracy and/or reduced error rates. In some embodiments, the nucleic acid barcodes are designed to have certain nucleotide sequences that make up overlapping dibase color positions (also called color positions). The order of the overlapping dibase color positions can be determined using fluorophore-encoded dibase probes in a fluorophore color calling scheme to give high fidelity reads.

This application claims the filing date benefit of U.S. Provisional Application Nos. 61/303,954, filed on Feb. 12, 2010; 61/307,348, filed on Feb. 23, 2010; 61/314,554, filed on Mar. 16, 2010; 61/356,491, filed on Jun. 18, 2010; and 61/391,574, filed on Oct. 8, 2010. The contents of each foregoing patent applications are incorporated by reference in their entirety.

### FIELD

The present teachings relate to identifier codes for use with, for example, nucleic acids, other biomolecules, or polymers, methods of designing and making codes, and methods of nucleic acid, biomolecule, or polymer sequencing using identifier codes.

### BACKGROUND

Upon completion of the Human Genome Project, one focus of the sequencing industry has shifted to finding higher throughput and/or lower cost sequencing technologies, sometimes referred to as “next generation” sequencing technologies. In making sequencing higher throughput and/or less expensive, the goal is to make the technology more accessible for sequencing. These goals can be reached through the use of sequencing platforms and methods that provide sample preparation for larger quantities of samples of significant complexity, sequencing larger numbers of complex samples, and/or a high volume of information generation and analysis in a short period of time. Various methods, such as, for example, sequencing by synthesis, sequencing by hybridization, and sequencing by ligation are evolving to meet these challenges.

To further increase throughput, it can also be desirable to sequence multiple samples at one time (referred to as multiplexed sequencing). For example, multiplexed sequencing can allow multiple samples, such as, for example, samples from different sources, to be analyzed in a single sequencing run (e.g., on a common slide or other sample holder platform) at the same time. When carrying out multiplexed sequencing, it can be desirable to be able to identify the source or identity of each sample.

To identify samples in multiplexed experiments, molecular barcodes have been developed. A molecular barcode is a uniquely identifiable marker attached to a sample nucleic acid. For example, a molecular barcode can comprise a short nucleic acid comprising a known sequence. A plurality of difference molecular barcodes can be used to identify samples belonging to a common group.

### SUMMARY

Provided herein are systems, compositions and methods for tracking, sorting and/or identifying sample nucleic acids, biomolecules, and polymers using identifiable codes. In some aspects, identifier codes can be designed to be uniquely identifiable. Identifier codes can be read, or otherwise recognized, identified, or interpreted as a function of a sequence or other arrangement or relationship of subunits that together form a code. In some exemplary embodiments, identifier codes can be read as a sequence of signals corresponding to the sequence or other arrangement or relationship of subunits that together form a code.

In some embodiments, identifier codes can be sequences of nucleotides, sets of nucleotides, biomolecule subunits, or polymer subunits. Identifier codes can correspond either directly or indirectly to or with sequences of nucleotides, sets of nucleotides, biomolecule subunits, or polymer subunits. For example, identifier codes can correspond to a sequence of individual nucleotides in a nucleic acid or subunits of a biomolecule or polymer or to sets, groups, or continuous or discontinuous sequences of multiple nucleotides or subunits. Identifier codes can also correspond to or with transitions between nucleotides, biomolecule subunits, or polymer subunits, or other relationships between subunits forming an identifier code.

Identifier codes can have properties that permit them to be read, or otherwise recognized, identified, or interpreted with improved accuracy and/or reduced error rates as compared to other identifier codes of comparable type, length, or complexity. In some embodiments, identifier codes can be designed as a set (which can include subsets) of individual identifier codes. In some embodiments, the identifier codes in a set, or in a subset, can be selected to adhere to certain criteria to improve accuracy and/or reduce error rates in reading, or otherwise recognizing, identifying, or interpreting the codes.

Identifier codes can also be designed to have properties that are useful for manipulating a nucleic acid, biomolecule, or polymer. Nucleic acid identifier codes can, in some embodiments, include restriction endonuclease recognition sequence or cleavage site, one or more overhang ends, adaptor sequences, one or more primer sequences, and the like (including combinations of features or properties). Biopolymer identifier codes can include, for example, antibody recognition sites, restriction sites, intra- or inert-molecule binding sites, and the like (including combinations of features or properties).

Also provided herein are libraries of nucleic acids, biomolecules, and polymers having identifier codes attached to or otherwise associated with them. Also provided are numerous exemplary identifier code sequences, set forth in SEQ ID. NOS 1-96, which can be used in a variety of sets, subsets, and groupings.

### DESCRIPTION OF THE DRAWINGS

FIG. 1 is a schematic depicting a non-limiting embodiment of a beaded template.

FIG. 2 is a schematic depicting a non-limiting embodiment of a beaded template.

FIG. 3 is a schematic depicting a non-limiting embodiment of a mate-pair beaded template.

FIG. 4A is a schematic depicting a non-limiting embodiment of a barcoded adaptor.

FIG. 4B is a schematic depicting a non-limiting embodiment of a beaded template.

FIG. 5 is a schematic depicting a non-limiting embodiment of a beaded template.

FIG. 6A is a list of color positions of barcodes 1-16 (top portion) and count of the color calls 0, 1, 2, and 3, (bottom portion) for non-limiting embodiments of nucleic acid barcodes.

FIG. 6B is a list of color positions of barcodes 1-16 (top portion) and count of the color calls 0, 1, 2, and 3, (bottom portion) for non-limiting embodiments of nucleic acid barcodes.

FIG. 7 is a list of nested color positions of barcodes 1-27 for non-limiting embodiments of nucleic acid barcodes.

FIGS. 8A and B are lists of barcoded adaptor sequences.

FIG. 9 is a list of universal complementary sequences.

FIGS. 10A and B are lists of sequencing primer sequences.

FIG. 11 is a schematic depicting a non-limiting embodiment of sequencing-by-ligation reactions.

It is to be understood that the figures are not drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DESCRIPTION OF VARIOUS EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As utilized in accordance with exemplary embodiments provided herein, the following terms, unless otherwise indicated, shall be understood to have the following meanings:

The phrase “next generation sequencing” refers to sequencing technologies having increased throughput compared to traditional Sanger- and capillary electrophoresis-based approaches, for example with the ability to generate hundreds of thousands of relatively short sequence read lengths at a time. Some examples of next generation sequencing techniques include, but are not limited to, sequencing by synthesis, sequencing by ligation, and sequencing by hybridization. Some relatively well-known next generations sequencing methods include pyrosequencing from 454 Corporation, Illumina's Solexa system, and the SOLiD™ (Sequencing by Oligonucleotide Ligation and Detection) from Applied Biosystems (now Life Technologies, Inc.).

The phrase “fragment library” refers to a collection of nucleic acid fragments, wherein one or more fragments are used as a sequencing template. A fragment library can be generated in numerous ways that are known in the art. As an example, a fragment library can be generated by cutting, shearing, restricting, or otherwise subdividing a larger nucleic acid into smaller fragments. Fragment libraries can be generated from naturally occurring nucleic acids, such as, for example, from bacteria, cancer cells, normal cells, or solid tissue. Libraries comprising synthetic nucleic acid sequences can also be generated to create a synthetic fragment library.

The phrase “mate pair library” refers to a collection of nucleic acid sequences comprising two or more fragments having a relationship, such as by being separated by a known number of nucleotides. Mate pair fragments can be generated in numerous ways that are known in the art. As an example, mate pair libraries can be generated by cutting, shearing, restricting, or otherwise subdividing a larger nucleic acid and associating the sequence fragments from the ends of the resulting fragments or by associating other subsequences of the resulting fragments. Mate pair libraries can be generated, for example, by circularizing a nucleic acid with an internal adapter construct and then removing the middle portion of the nucleic acid to create a linear strand of nucleic acid comprising the internal adapter with the sequences from the ends of the nucleic acid attached to either end of the internal adapter. Like fragment libraries, mate-pair libraries can be generated from naturally occurring nucleic acid sequences, such as for example, from bacteria, cancer cells, normal cells, or solid tissue. Synthetic mate-pair libraries can also be generated by attaching synthetic nucleic acid sequences to either end of an internal adapter sequence.

The phrase “synthetic nucleic acid sequence” and variations thereof refers to a designed and synthesized sequence of nucleic acid. For example, a synthetic nucleic acid sequence can be designed to follow rules or guidelines.

The term “template” and variations thereof refer to a nucleic acid sequence that is a target of nucleic acid sequencing reactions. A template sequence can comprise a naturally-occurring or synthetic nucleic acid sequence. A template sequence also can include a known or unknown nucleic acid sequence from a sample of interest. In various exemplary embodiments herein, a template sequence can be attached to a solid support, such as, for example, a bead, microparticle, flow cell, or any other surface or object.

The phrase “identifier codes” refer to compositions that can be used for tracking, sorting and/or identifying sample nucleic acids, biomolecules, and polymers. Identifier codes can be read, or otherwise recognized, identified, or interpreted as a function of a sequence or other arrangement or relationship of subunits that together form a code. Identifier codes can be comprised of the same kind or type of material or subunits comprising the nucleic acid, biomolecule, or polymer, or of a different material or subunit. Although identifier codes are exemplified herein in the context of nucleic acid sequences, they are not limited to that context or set of embodiments and the teachings herein are applicable to identifier codes for use with biomolecules and polymers.

The phrases “nucleic acid barcode”, “barcode”, and variations refer to an identifiable nucleotide sequence, such as an oligonucleotide or polynucleotide sequence. In some embodiments, nucleic acid barcodes are uniquely identifiable. Provided herein is a system, comprising a plurality of identifiable nucleic acid barcodes. In some embodiments, nucleic acid barcodes can be attached to, or associated with, target nucleic acid fragments to form barcoded target fragments. A library of barcoded target fragments can include a plurality of a first barcode attached to target fragments from a first source. Alternatively, a library of barcoded target fragments can include different identifiable barcodes attached to target fragments from different sources to make a multiplex library. For example, a multiplex library can include a mixture of a plurality of a first barcode attached to target fragments from a first source, and a plurality of a second barcode attached to target fragments from a second source. In the multiplex library, the first and second barcodes can be used to identify the source of the first and second target fragments, respectively. The skilled artisan will appreciate that any number of different barcodes can be attached to target fragments from any number of different sources. In a library of barcoded target fragments, the barcode portion can be used to identify: a single target fragment; a single source of the target fragments; a group of target fragments; target fragments from a single source; target fragments from different sources; target fragments from a user-defined group; or any other grouping that requires identification. The sequence of the barcoded portion of the barcoded target fragment can be separately read from the target fragment, or read as part of a larger read spanning the barcode and the target fragment. In a sequencing experiment, the nucleic acid barcode can be sequenced with the target fragment and then parsed algorithmically during processing of the sequencing data. In some embodiments, a nucleic acid barcode can comprise a synthetic or natural nucleic acid sequence, DNA, RNA, or other nucleic acids and/or derivatives. For example, a nucleic acid barcode can include nucleotide bases adenine, guanine, cytosine, thymine, uracil, inosine, or analogs thereof.

### Fidelity

Provided herein are nucleic acid barcodes designed to exhibit high fidelity sequencing reads. In some embodiments, the level of fidelity can be based on empirical measurements of the barcode in a sequencing reaction. In some embodiments, the level of fidelity can be based on predictions of the read accuracy of a barcode having a particular nucleotide sequence. For example, certain nucleotide sequences known to cause sequencing read errors can be avoided, or certain nucleotide sequence known to give sequencing bias can be avoided. In some embodiments, the design of the barcodes can be based on accurately calling the correct color of a fluorophore-labeled nucleotide or fluorophore-labeled probe used for the sequencing reaction. For example, the barcodes can be based on accurate color calling in a base space or a color space sequencing system. In some embodiments, in a color space system, the barcodes can be designed to exhibit color balance, 3-different color positions, or nested color call sequences. In some embodiments, the probability of correctly determining the sequence of the nucleic acid barcodes can be at least 82%, or at least 85%, or at least 90%, or at least 95%, or at least 99%, or higher fidelity.

### Forbidden Sequences

Provided herein are nucleic acid barcodes designed to avoid base sequences that may be problematic. For example, repetitive sequences can be avoided, such as 5 -GGGG-′3 and 5′-CCCC-3′. Other sequences that can be avoided include those that result in repetitive color calls. For example, sequences that result in the same color call 4 or more times can be avoided (Table 1). Other sequences that can be avoided include A-T rich and G-C rich sequences, such as, for example, {A,T}5 and {G,C}5.

### Sequencing the Barcodes in Base Space

In some embodiments, the nucleic acid barcodes are designed to exhibit improved read accuracy for sequencing in a base space system (e.g., sequence-by-synthesis systems). In some embodiments, the barcoded libraries can be sequenced in base space, using fluorophore-labeled nucleotides and one or more template-dependent DNA polymerases which polymerize the labeled nucleotides. The sequence of the templates can be determined by correlating a one-to-one relationship of an incorporated labeled nucleotide and the template nucleotide. Examples of base-space sequencing include capillary electrophoresis (Applied Biosystems), pyrophosphate sequencing system by 454, and Solexa sequencing system by Illumina.

In some embodiments, identifier codes can be read, identified, interpreted or otherwise recognized using methods known in the art, including for example amino acid sequencing for protein identifier codes.

### Color Space

In some embodiments, the nucleic acid barcodes are designed to exhibit improved read accuracy for sequencing in a color space system. In some embodiments, in a color space system, the nucleic acid barcodes comprise a nucleotide sequence that forms overlapping dibase color positions. The order of the overlapping dibase color positions can be determined by fluorophore color calling using a 2-base degenerate color call system.

TABLE 1

Dye Y

| | | | | |
|---|---|---|---|---|
| Dye (XY) | A | C | G | T |

| | | | | | |
|---|---|---|---|---|---|
| Dye X | A | 0 | 1 | 2 | 3 |
| | C | 1 | 0 | 3 | 2 |
| | G | 2 | 3 | 0 | 1 |
| | T | 3 | 2 | 1 | 0 |

```
(SEQ ID NO: 139)
5′-G C C T C T T A C A C-3′
        3 0 2 2 2 0 3 1 1 1
    -G C N N N N N N-
        3
        -C C N N N N N N-
            0
            -C T N N N N N N-
                2
                -T C N N N N N N-
                    2
                    -C T N N N N N N-
                        2
                        -T T N N N N N N-
                            0
                            -T A N N N N N N-
                                3
                                -A C N N N N N N-
                                    1
                                    -C A N N N N N N-
                                        1
                                        -A C N N N N N N-
                                            1
```

The schematic above, and Table 1, show one embodiment of a color calling scheme. A nucleic acid barcode is an oligonucleotide where the order of the bases in the barcode make up overlapping dibase color positions, also called color positions.

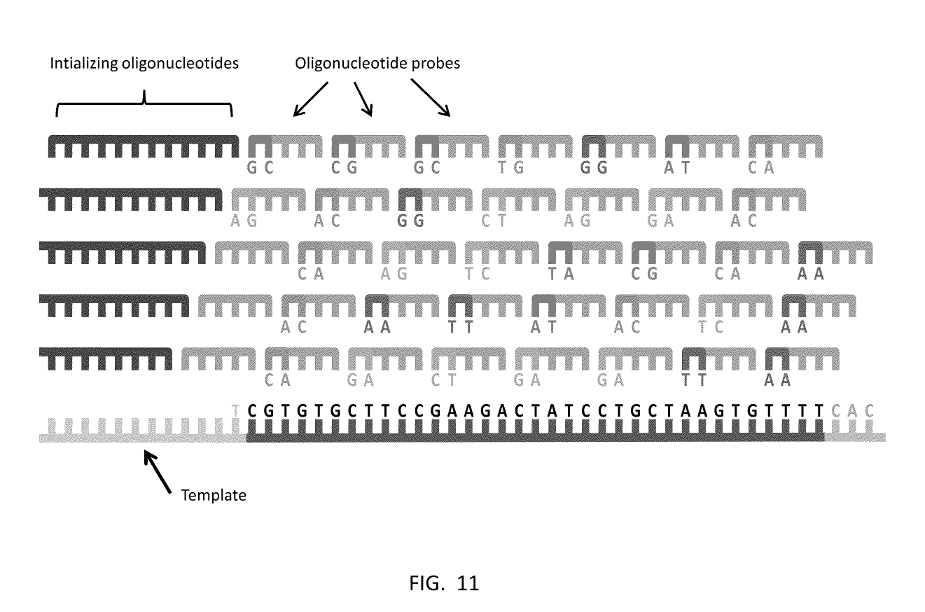

In some embodiments, a nucleic acid barcode can be sequenced in a color space using fluorophore-encoded dibase probes that hybridize to the barcode template. In some embodiments, the probes are complementary to the barcode template. In the example shown above, the dibase probes are 8-mers, where the first two bases are encoded by one of four fluorophores (fluorophore-encoded) which are designated 0, 1, 2, or 3. The letter “N” denotes any base. In some embodiment, the color calling step includes identifying the color of the fluorophore-encoded dibase probe that is hybridized to the barcode template, using the decoding Table 1. In successive cycles, fluorophore-encoded dibase probes hybridize to the barcode template, and the color of the fluorophore-labeled probe is identified (FIG. 11). In the example shown above, the color call “2” is in the third, fourth, and fifth color position of the barcode. It will be readily appreciated by the skilled artisan that other decoding color calling schemes, other than that shown in Table 1, can be used.

Provided herein is a system, comprising a plurality of identifiable nucleic acid barcodes comprising overlapping dibase color positions. In some embodiments of the system, the overlapping dibase color positions can be sequenced in a color space. In some embodiments of the system, the sequence of the color positions can be determined using fluorophore encoded dibase probes. At least two, three, four, or more fluorophore encoded dibase probes can be used to determine the sequence of the color positions. In some embodiments of the system, in successive cycles, the fluorophore-encoded dibase probes hybridize to the barcode template, and the color of the fluorophore-labeled probe is identified.

Provided herein is a method for sequencing a nucleic acid barcode, comprising successively hybridizing a nucleic acid barcode with a fluorophore-encoded dibase probe and identifying the color of the fluorophore-encoded dibase probe, so hybridized. The colors of the fluorophore-encoded dibase probe that are identified in the successive hybridization cycles are not sufficient to determine the base sequence of the barcode, without additional information. For example, identifying other bases of the barcode, in addition to identifying the colors of the fluorophore-encoded dibase probe that are identified in the successive hybridization cycles may be sufficient to determine the sequence of the nucleic acid barcode.

An example of color space sequencing includes SOLiD™ sequencing systems (e.g., WO 2006/084132) by Applied Biosystems (now part of Life Technologies, Carlsbad, Calif.). However, as one skilled in the art would readily appreciate, the nucleic acid barcodes, and methods for designing the barcodes described herein can be applied to other sequencing systems or detection techniques, including but not limited to, for example, other next generating sequencing systems and detection techniques. The principles of nucleic acid barcodes and methods using the nucleic acid barcodes can be applied to other systems and methods without departing from the scope of the present teachings as described herein.

Other exemplary embodiments of the present teachings relate to designing nucleic acid barcodes combined with yeast barcodes. Various exemplary embodiments relate to methods for sequencing yeast gene deletion sequences using nucleic acid barcodes.

### Examples of Color Calling

In some embodiments, the dibase fluorophores color calling sequencing system includes 4 color calls (e.g., 4 fluorescent-detectable dye colors) which are available for the 16 possible 2-base combinations. Therefore, it is possible that different sequences may yield the same color calls. For example, 5′-AAAAA-3′ may have the same color call of “0” as 5′-TTTTT-3′, 5′-CCCCC-3′, and 5′-GGGGG-3′ (see Table I). Thus, the number of uniquely identifiable nucleic acid barcode sequences available is not equal to the number of possible nucleotide sequences for a given length. For example, in the simplest scenario of a 2-base nucleic acid barcode, of the 16 possible combinations of 2 nucleotides, only 4 unique color calls are observable and therefore a maximum of 4 uniquely identifiable barcodes would be available.

In some embodiments, a nucleic acid barcode can be attached to a sample having a terminal base A, T, G, C, or any nucleotide analog. Thus, a 10-mer barcode having the sequence CCTCTTACAC (SEQ ID NO:1) and attached to a sample having a terminal base G, will give a dibase color call as follows:

| 5′-G C C T C T T A C A C-3′ | (SEQ ID NO: 139) |
|---|---|
| 3 0 2 2 2 0 3 1 1 1 | |

In the example shown above, the first nucleotide (e.g., G) is not part of the barcodes sequence, but is part of the nucleic acid sample sequence that is ligated to the barcode. For example, in the example shown above, the color call “2” is in third, fourth, and fifth color position.

### Color Balance

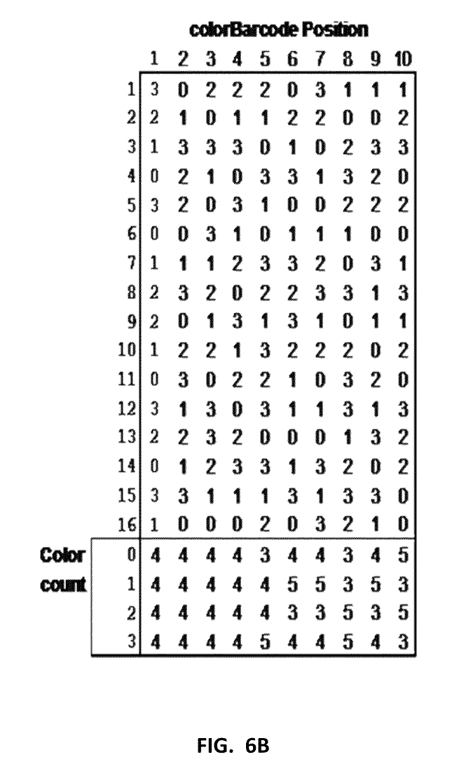

In some embodiments, nucleic acid barcodes, or a set of barcodes, can be designed to be color balanced. In some embodiments, a set of nucleic acid barcodes can be color balanced in all positions or in a subset of positions. For example, a set of barcodes can include four 10-mer barcodes (e.g., 24 sets of 4 barcodes for a total of 96 barcodes). A set of four barcodes can be designed to have all four colors (e.g., 0, 1, 2, and 3) represented in all 10 positions across the set (see FIGS. 6A and B). FIG. 6A shows barcodes that are not color balanced, because the color “0” (zero) does not appear in the sixth position in any barcode. However, FIG. 6B shows barcodes that are color balanced because, as a set of 16 barcodes, the colors 0, 1, 2 and 3 are represented in all 10 positions.

### 3-Different Color Positions

In some embodiments, the nucleic acid barcodes can be designed to have nucleotide sequences that, in a color call system, any two barcodes will differ in at least 3 color positions. In the example shown below, a comparison of barcodes 1 and 20 show that they differ in their color call at positions 3, 4 and 5 (underlined and bolded).

BC 1: 3022203111

BC 20: 3001303111

### Empirical Performance

In some embodiments, the nucleic acid barcode can be designed to optimize the barcode's observed performance in a sequencing process. A Constraint Satisfaction Algorithm can be used to design the barcodes based on desired properties. Design criteria that can improve the observed nucleic acid barcode performance include, but are not limited to the uniqueness of the nucleic acid barcode sequences, the degree of separation from other nucleic acid barcode sequences, and color balance during sequencing. According to various embodiments, one or more of these criteria can be used to design the nucleic acid barcode.

### Nested Sequences

In some embodiments, a set of nucleic acid barcodes can be a nested set of barcodes which include one or more of the design criteria described above. Nested barcode sets can be described as analogous to Matryoshka nesting wherein the properties of a subset are entirely contained within the properties of a genus set. For example, a first subset of nucleic acid barcodes, which can be color balanced and exhibit high sequencing fidelity, can be selected from a larger set of nucleic acid barcodes, which is also color balanced and exhibits high sequencing fidelity. In at least one embodiment, a full set of nucleic acid barcodes can comprise 96 uniquely identifiable barcodes. If a sequencing experiment comprises only 16 multiplexed samples, a subset of 16 nucleic acid barcodes can be selected from the 96 available barcodes. The subset of 16 nucleic acid barcodes can thus be optimized to a similar degree as a larger subset of 32 nucleic acid barcodes or 48 nucleic acid barcodes selected from the full set of 96 nucleic acid barcodes.

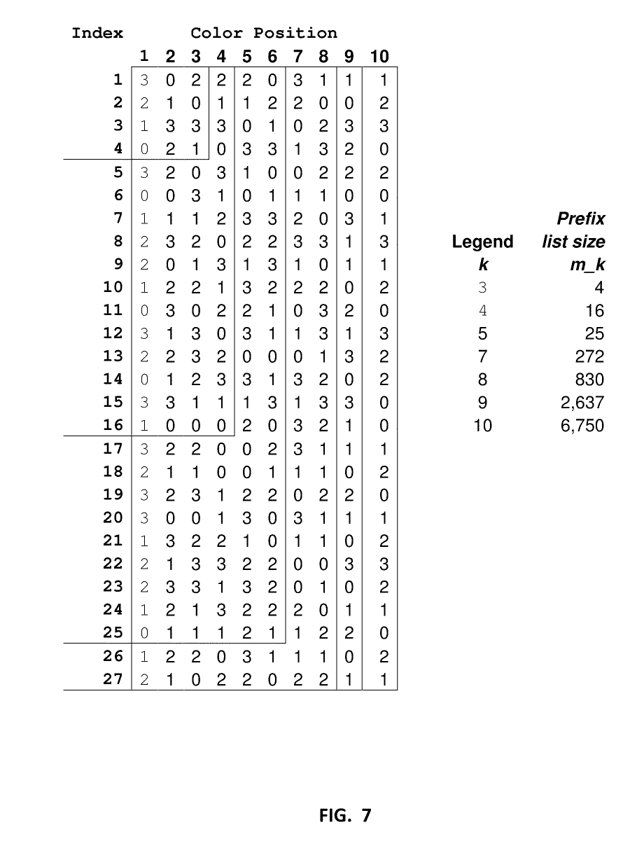

In some embodiments, the nucleic acid barcodes can be designed as an ordered list of nested barcodes. In some embodiments, when taken in order, as many barcodes as possible have different colors in all 3 positions in the first 3 positions of the barcode (see FIG. 7). In some embodiments, when taken in order, as many barcodes as possible have different colors in all 3 positions in the first 4 positions of the barcode (for k=4). In some embodiments, when taken in order, as many barcodes as possible have different colors in all 3 positions in the first 5 positions of the barcode (for k=5).

### Length

The length of the nucleic acid barcodes can be any length, such as for example 4-30 base, or 4-50 bases, or more. In some embodiments, the length of the barcode can be based on the length of the fluorophore-encoded dibase probes used during color space sequencing. For example, if the probe sequence ligated during each ligation cycle of a sequencing experiment (for example, a SOLiD™ sequencing experiment) is 5 bases, the nucleic acid barcode can have a length that is a multiple of 5, such as, for example, 5 bases, 10 bases, 15 bases, etc. Similarly, if the probe sequence ligated during each ligation cycle is 4 bases, the nucleic acid barcode can have a length that is a multiple of 4, such as, for example, 4, 8, 12, etc. bases. If the probe sequence ligated during each ligation cycle is 6 bases, the nucleic acid barcode can have a length that is a multiple of 6, such as, for example, 6, 12, 18, etc. bases. When sequencing by ligation, as in the SOLiD system, this “multiples” relationship can ensure that the sequencing of the barcode is completed after the same number of ligation cycles as is the sequencing of the template sequence.

In some embodiments, the length of the nucleic acid barcodes can be selected based on the number of samples for which unique identification may be desired. Due to the number of possible variations of nucleotides in a nucleic acid sequence, the nucleic acid barcode can have a length that is selected based on the number of samples. For example, in a 16 sample multiplexed sequencing experiment, 16 uniquely identifiable nucleic acid barcodes would be sufficient to uniquely identify each sample. Similarly, a 64- or 96-sample multiplexed sequencing experiment can utilize 64 or 96 uniquely identifiable nucleic acid barcodes, respectively.

In some embodiments, the length of the nucleic acid barcode can be selected based on both the length of the probe sequence and the number of samples in the multiplexed sequencing experiment. As above, the length of the barcode can be selected as a multiple of the probe sequence length. In addition, the length of the barcode can be longer for a larger number of samples. For example, in a 16-sample multiplexed sequencing experiment using 5-base probe sequences, the nucleic acid barcode can be 5 bases in length. In a 96-sample multiplexed sequencing experiment using 5-base probe sequences, the nucleic acid barcode can be 10 bases.

### Combination of Criterion

In some embodiments, a set of nucleic acid barcodes can be designed based on at least one of the criteria set forth above, or based on any combination of the criteria set forth above. For example, a set of nucleic acid barcodes can be designed such that problematic sequences are avoided and color balance is achieved in all positions. In another example, a set of nucleic acid barcodes can be designed such that problematic sequences are avoided, color balance is achieved in all positions, and the nucleic acid barcodes are sequenced with high fidelity. Other combinations of the design criteria may be chosen based on the sequencing experiment being run. For example, if a set of nucleic acid barcodes is used for a small number of multiplexed samples, the set of nucleic acid barcodes would not necessarily be designed to have nested subsets. In another example, if a large number of multiplexed samples are being analyzed, the set of nucleic acid barcodes might not be color balanced in all positions. One of ordinary skill in the art would recognize that the design criteria can be selected based on the number of samples being analyzed, the required accuracy needed, the sensitivity of the sequencing instrument to detect individual samples, the accuracy of the sequencing instrument, etc. Nucleic acid barcodes having at least some of these properties need not be sequenced to the 10^{th} position for barcode identity.

Referring to Table 2 below, an exemplary set of 96 nucleic acid barcodes of 10 bases in length is shown. The set of nucleic acid barcodes shown in Table 2 can be used, for example, in a multiplexed dibase sequencing experiment with up to 96 different samples.

TABLE 2

| | | |
|---|---|---|
| 1 | CCTCTTACAC | SEQ ID NO. 1 |
| 2 | ACCACTCCCT | SEQ ID NO. 2 |
| 3 | TATAACCTAT | SEQ ID NO. 3 |
| 4 | GACCGCATCC | SEQ ID NO. 4 |
| 5 | CTTACACCAC | SEQ ID NO. 5 |
| 6 | TGTCCCTCGC | SEQ ID NO. 6 |
| 7 | GGCATAACCC | SEQ ID NO. 7 |
| 8 | ATCCTCGCTC | SEQ ID NO. 8 |
| 9 | GTCGCAACCT | SEQ ID NO. 9 |
| 10 | AGCTTACCGC | SEQ ID NO. 10 |
| 11 | CGTGTCGCAC | SEQ ID NO. 11 |
| 12 | TTTTCCTCTT | SEQ ID NO. 12 |
| 13 | GCCTTACCGC | SEQ ID NO. 13 |
| 14 | TCTGCCGCAC | SEQ ID NO. 14 |
| 15 | CATTCAACTC | SEQ ID NO. 15 |
| 16 | AACGTCTCCC | SEQ ID NO. 16 |
| 17 | GCGGTGAGCC | SEQ ID NO. 17 |
| 18 | TCATCCGCCT | SEQ ID NO. 18 |
| 19 | CAGTTACCAT | SEQ ID NO. 19 |
| 20 | AAAGCTTGAC | SEQ ID NO. 20 |
| 21 | GGAACCGCAC | SEQ ID NO. 21 |
| 22 | TCATCTTCTC | SEQ ID NO. 22 |
| 23 | CAAGCACCGC | SEQ ID NO. 23 |
| 24 | ATACCGACCC | SEQ ID NO. 24 |
| 25 | TCATCATGTT | SEQ ID NO. 25 |
| 26 | CGGGCTCCCG | SEQ ID NO. 26 |
| 27 | AAGTTTGCTG | SEQ ID NO. 27 |
| 28 | GTAGTAAGCT | SEQ ID NO. 28 |
| 29 | CCCTAGATTC | SEQ ID NO. 29 |
| 30 | TCTTCGCTAC | SEQ ID NO. 30 |
| 31 | ACGCACCAGC | SEQ ID NO. 31 |
| 32 | GCACCCAACC | SEQ ID NO. 32 |
| 33 | GTATCCAACG | SEQ ID NO. 33 |
| 34 | CCTTTAACGA | SEQ ID NO. 34 |
| 35 | TCCTACGCTT | SEQ ID NO. 35 |
| 36 | ATGTGAGAAC | SEQ ID NO. 36 |
| 37 | GGTATAACAG | SEQ ID NO. 37 |
| 38 | CTAAGACGAC | SEQ ID NO. 38 |
| 39 | ACTCACGATA | SEQ ID NO. 39 |
| 40 | TAACCCTTTT | SEQ ID NO. 40 |
| 41 | CAATCCCACA | SEQ ID NO. 41 |
| 42 | TAGTACATTC | SEQ ID NO. 42 |
| 43 | AACCCTAGCG | SEQ ID NO. 43 |
| 44 | GATCATCCTT | SEQ ID NO. 44 |
| 45 | AGCCAAGTAC | SEQ ID NO. 45 |
| 46 | TTCGACGACC | SEQ ID NO. 46 |
| 47 | GCCATCCCTC | SEQ ID NO. 47 |
| 48 | CACTTACGGC | SEQ ID NO. 48 |
| 49 | CTTATGACAT | SEQ ID NO. 49 |
| 50 | GCAAGCCTTC | SEQ ID NO. 50 |
| 51 | ACTCCTGCTT | SEQ ID NO. 51 |
| 52 | TTACAATTAC | SEQ ID NO. 52 |
| 53 | ACTTGATGAC | SEQ ID NO. 53 |
| 54 | TCCGCCTTTT | SEQ ID NO. 54 |
| 55 | CGCTTAAGCT | SEQ ID NO. 55 |
| 56 | GGTGACATGC | SEQ ID NO. 56 |
| 57 | TTCTTACTAG | SEQ ID NO. 57 |
| 58 | CGCCACTTTA | SEQ ID NO. 58 |
| 59 | GACATTACTT | SEQ ID NO. 59 |
| 60 | ACCGAGGCAC | SEQ ID NO. 60 |
| 61 | CGATAATCTT | SEQ ID NO. 61 |
| 62 | ACCCTCACCT | SEQ ID NO. 62 |
| 63 | TCGAACCCGC | SEQ ID NO. 63 |
| 64 | GGTGTAGCAC | SEQ ID NO. 64 |
| 65 | GCTTGATCCC | SEQ ID NO. 65 |
| 66 | ACATTACATC | SEQ ID NO. 66 |
| 67 | CCCTAAGGAC | SEQ ID NO. 67 |
| 68 | TCGTCAATGC | SEQ ID NO. 68 |
| 69 | AAAGCATATC | SEQ ID NO. 69 |
| 70 | TCTGTAGGGC | SEQ ID NO. 70 |
| 71 | CGTTCCCTGT | SEQ ID NO. 71 |
| 72 | GTATTCACTT | SEQ ID NO. 72 |
| 73 | ACGTCATTGC | SEQ ID NO. 73 |
| 74 | TCAGCGTCCT | SEQ ID NO. 74 |
| 75 | GCCCAGATAC | SEQ ID NO. 75 |
| 76 | CCTAAAACTT | SEQ ID NO. 76 |
| 77 | AAGACCAGAT | SEQ ID NO. 77 |
| 78 | GATGATTGCC | SEQ ID NO. 78 |
| 79 | TAATTCTACT | SEQ ID NO. 79 |
| 80 | CACCGTAAAC | SEQ ID NO. 80 |
| 81 | AATGACGTTC | SEQ ID NO. 81 |
| 82 | CTCCCTTCAC | SEQ ID NO. 82 |
| 83 | TACGCCATCC | SEQ ID NO. 83 |
| 84 | GTTCATCCGC | SEQ ID NO. 84 |
| 85 | AACGCTTTCC | SEQ ID NO. 85 |
| 86 | TCCTGGTACT | SEQ ID NO. 86 |
| 87 | GCTTTGCTAT | SEQ ID NO. 87 |
| 88 | CATGATCAAC | SEQ ID NO. 88 |
| 89 | TAGACAGCCT | SEQ ID NO. 89 |
| 90 | AGTAGGTCAC | SEQ ID NO. 90 |
| 91 | CCCAATACGC | SEQ ID NO. 91 |
| 92 | GTAATCCCTT | SEQ ID NO. 92 |
| 93 | GCATCGTAAC | SEQ ID NO. 93 |
| 94 | AAACACCCAT | SEQ ID NO. 94 |
| 95 | TGCCGGACTC | SEQ ID NO. 95 |
| 96 | CTCTTCGATT | SEQ ID NO. 96 |

### Multiplex Libraries

Provided herein are nucleic acid barcodes that can be attached to, or associated with, target nucleic acid fragments to generate barcoded nucleic acid libraries.

The barcoded nucleic acid libraries can be prepared using any known nucleic acid manipulation procedure in any combination and in any order, including: fragmenting; size-selecting; end-repairing; tailing; adaptor-joining; nick translation; and purification.

In some embodiments, the nucleic acid barcodes can be attached to, or associated with, the fragments of the target nucleic acid sample using any art known procedure, including ligation, cohesive-end hybridization, nick-translation, primer extension, or amplification. In some embodiments, the nucleic acid barcodes can be attached to the target nucleic acid using amplification primers having the barcode sequence.

### Target Nucleic Acids

In some embodiments, the target nucleic acid sample can be isolated from any source, such as solid tissue, tissue, cells, yeast, bacteria, or similar sources of nucleic acid samples. Methods for isolating nucleic acids from these sources are well known in the art. For example, the solid tissue or tissue can be weighed, cut, mashed, homogenized, and the nucleic acid can be isolated from the homogenized samples. The isolated nucleic acids can be chromatin which can be cross-linked with proteins that bind DNA, in a procedure known as ChIP (chromatin immunoprecipitation).

In some embodiments, the biomolecules include polymers such as proteins, polysaccharides, and nucleic acids, and their polymer subunits. The biomolecules can be isolated from any source such as solid tissue, tissue, cells, yeast, or bacteria. Methods for isolating biomolecules from these sources are well known in the art. For example, the solid tissue or tissue can be weighed, cut, mashed, homogenized, and the biomolecules can be isolated from the homogenized samples.

In some embodiments, the target nucleic acid sample can be fragmented to prepare target nucleic acid fragments, using any procedure known in the art, including cleaving with and enzyme or chemical, or by shearing. Enzyme cleavage includes any type of restriction endonuclease, endonuclease, or transposase-mediated cleavage. In some embodiments, the biomolecules can be fragmented using well known methods, including enzymatic or chemical cleavage, or shearing forces.

### Fragment Libraries

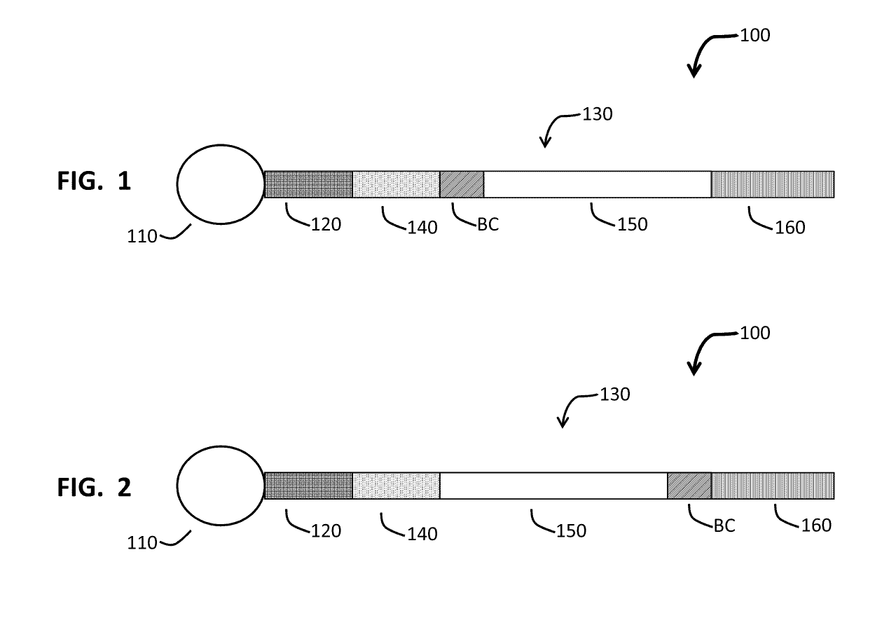

Provided herein are fragment libraries, comprising a first priming site (P1), a second priming site (P2), an insert, an internal adaptor (IA), and a barcode (BC). In some embodiments, the fragment library can include constructs having certain arrangements, such as: P1 priming site, insert, internal adaptor (IA), barcode (BC), and P2 priming site. In some embodiments, the fragment library can be attached to solid support, such as beads. An exemplary nucleic acid attached to a solid support, such as a bead, for use in sequencing by ligation is shown in FIG. 1. As depicted in FIG. 1, various embodiments of beaded template 100 include a bead 110 having a linker 120, which is a sequence for attaching a template 130 to the solid support. The template 130 can include a first or P1 priming site 140, an insert 150, and a second or P2 priming site 160. In one embodiment, an internal adaptor can be placed between the P1 priming site 140 and the barcode BC, or between the barcode BC and insert 150, or between the insert 150 and P2 priming site 160. The length of each of the linker 120 and synthetic template 130 can vary. For example, the length of the linker 120 can range from 10 to 100 bases, for example, from 15 to 45 bases, such as, for example, 18 bases (18b) in length. Template 130, which comprises P1 140, insert 150, and P2 160, can also vary in length. In at least one embodiment, P1 140 and P2 160 can each range from 10 to 100 bases, for example, from 15 to 45 bases, such as, for example, 23 bases (23b) in length. The insert 150 can range from 2 bases (2b) to 20,000 bases (20 kb), such as, for example, 60 bases (60b). In at least one embodiment, the insert 150 can comprise more than 100 bases, such as, for example, 1,000 or more bases. In various embodiments, the insert can be in the form of a concatenate, in which case, the insert 150 can comprise up to 100,000 bases (100 kb) or more.

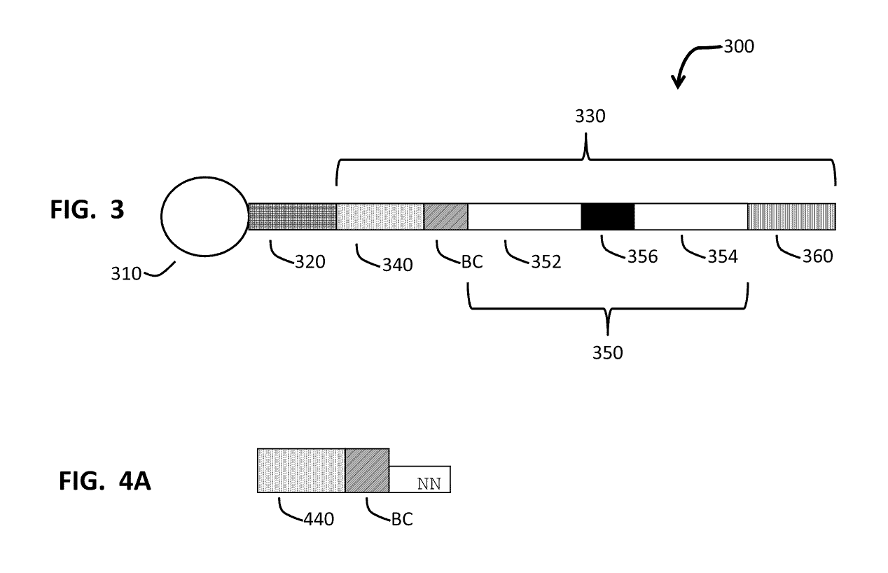

In some embodiments, template 130 can further comprise a nucleic acid barcode BC. In FIG. 1, nucleic acid barcode BC is positioned between primer P1 140 and the insert 150. In another embodiment, nucleic acid barcode BC can be positioned between insert 150 and primer P2 160, as shown in the exemplary embodiment of FIG. 2. In one embodiment, an internal adaptor can be placed between the P1 priming site 140 and the insert 150, or between the insert 150 and the barcode BC, or between the barcode BC and the P2 priming site 160. A person of ordinary skill would recognize other locations for the bar code in other embodiments.

In some embodiments, the position of nucleic acid barcode BC can be selected based on the length of the insert and/or to avoid any potential sequencing bias. For example, the signal to noise ratio can decrease as additional ligation cycles are performed. When signal to noise may be an issue, the nucleic acid barcode BC can be positioned adjacent primer P1 140 to avoid potential errors due to diminished signal to noise. In situations where the signal to noise ratio may not vary significantly from early ligation cycles to later ligation cycles, the nucleic acid barcode BC can be placed adjacent to either primer P1 140 or primer P2 160.

In some embodiments, the position of nucleic acid barcode BC can be selected to avoid potential sequencing bias. For example, some template sequences may interact differently with a probe sequence used during the sequencing experiment. Placing the nucleic acid barcode BC before the insert 150 can affect the sequencing results for the insert 150. Positioning the nucleic acid barcode BC after the insert 150 can decrease sequencing errors due to bias. One of ordinary skill in the art would recognize that the position of the nucleic acid barcode BC can be affected by or affect the sequencing process and accordingly can chose the position that best achieves the desired results based on the conditions of the sequencing process.

For sequencing and decoding of the nucleic acid barcode BC, a single forward direction sequence read can be performed (e.g., 5′-3′ direction along the template) (e.g., F3/tag1), reading both the barcode BC and the insert 150 in a single read. The forward read can be parsed into the barcode portion and the insert portion algorithmically.

In some embodiments, identifier codes can be attached to polymers such as proteins. In some embodiments, the identifier codes can be polypeptides that are attached to a protein. In some embodiments, intein-mediated ligation can join together separate proteins or polypeptides. For example, expressed protein ligation (EPL) involves a native chemical ligation (NCL) reaction between an intein-fusion protein and protein having an N-Cys. In another example, protein trans-splicing involves reconstitution of two halves of an intein protein (Dawson 1994 Science 266:776-779; Muir 2003 Ann. Rev. Biochem. 72:249-289; Paulus 2000 Ann. Rev. Biochem. 69:447-496; and Muralidharan 2006 Nature Methods 3:429-438).

### Mate Pair Libraries

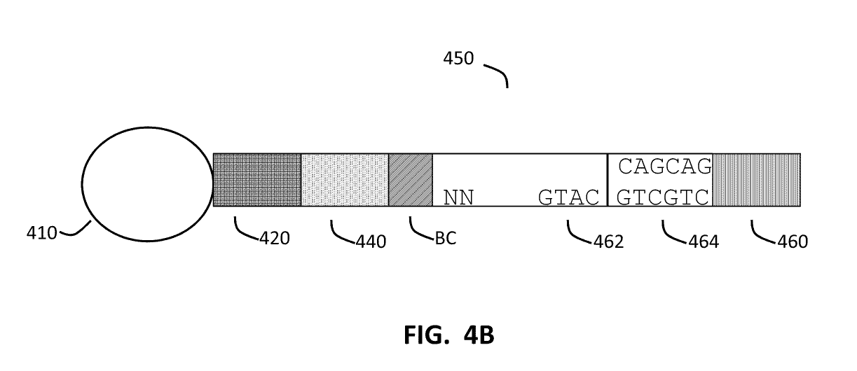

FIG. 1 and FIG. 2 depict a template 130 representative of a fragment library. The nucleic acid barcodes of the present teachings can also be used in templates derived from a mate-pair library. FIG. 3 schematically depicts a beaded template 300 comprising a bead 310, a linker 320, and a template 330. The template 330 of synthetic bead 300 can be analogous to a mate pair library construction. Template 330 can comprise a first or P1 priming site 340 and second or P2 priming site 360, each of which can range in length from 10 to 100 bases, for example, from 15 to 45 bases, such as, for example, 23 bases in length. Template 330 further comprises an insert 350, which can comprise a first tag sequence 352, a second tag sequence 354, and an internal adapter 356 located between the first and second tag sequences 352, 354. In some embodiments, the barcode BC can be placed between the second tag sequence 354 and the P2 priming site 360. One skilled in the art will recognize other positions to place the barcode BC. The first and second tag sequences 352, 354 can each have a length ranging from 2 bases (2b) to 20,000 bases (20 kb), such as, for example, 60 bases. The first and second tag sequences 352, 354 can be the same sequence or different sequences. The first and second tag sequences 352, 354 can comprise a different number of bases or the same number of bases. The internal adapter 356, which can be common to all of the template sequences, can have a length ranging from 10 to 100 bases, for example, from 15 to 45 bases, such as, for example, 36 bases.

In some embodiments, the nucleic acid barcode can be incorporated into an extended oligonucleotide comprising the nucleic acid barcode and one or more sequences including the P1 primer, the P2 primer, and an internal adapter. For example, in at least one embodiment, the nucleic acid barcode can be incorporated into an oligonucleotide comprising the P2 primer, the nucleic acid barcode, and an internal adapter, which can allow the nucleic acid barcode to be sequenced in a separate read. One skilled in the art would recognize that the nucleic acid barcode can be incorporated into other oligonucleotides or arrangements of oligonucleotides without departing from the scope of the present teachings.

In FIG. 3, a nucleic acid barcode BC is positioned between primer P1 340 and first tag sequence 352. As described above, however, the position of nucleic acid barcode BC can be chosen based on the conditions of the sequencing process. For example, the nucleic acid barcode BC can be positioned between primer P1 340 and a first tag sequence 352, as shown in FIG. 3, or the nucleic acid barcode BC can be positioned between a second tag sequence 354 and the primer P2 360. Alternatively, nucleic acid barcode BC can be positioned adjacent an internal adapter 356 and either first tag sequence 352 or second tag sequence 354. In another embodiment, the barcode BC can be integrated within an internal adapter 356.

Nucleic acid barcodes in accordance with various exemplary embodiments of the present teachings can be added to libraries using any known method. For example, full-length double-stranded oligonucleotide pairs specific for each nucleic acid barcode can be annealed and ligated onto double-stranded nucleic acid fragments. In another example, one full-length double-stranded oligonucleotide can be annealed to one short universal oligonucleotide specific for each barcode and ligated onto double-stranded nucleic acid fragments. In a further example, a universal oligonucleotide adapter can be ligated onto single-stranded RNA, converted into double-stranded DNA, then the nucleic acid barcode can be added using a barcode-specific PCR primer during library amplification.

The nucleic acid barcodes can be adapted for use in generating mate pair libraries for nucleic acid sequencing. For example, the nucleic acid barcodes can be used in the SOLiD™ Mate-Paired Library Construction Kits developed by Applied Biosystems (now Life Technologies, Inc.). In some embodiments, the P2 adaptor can be replaced with a multiplex adaptor having three portions: an internal primer binding sequence; a barcode sequence; and a P2 primer binding sequence.

As shown in FIG. 3, such mate pair constructs can comprise a template 330 with a first or P1 priming site 340 and second or P2 priming site 360. The template 330 further comprises an insert 350, which can comprise a first sheared DNA tag sequence 352, a second sheared DNA tag sequence 354, and an internal adaptor 356 located between the first and second sheared tag sequences 352, 354. Because the internal adaptor sequence is located in between the two tag sequences 352, 354, an alternative sequence can be used to prime the sequencing of the barcode BC as disclosed herein.

To construct barcoded mate pair libraries using nucleic acid barcodes positioned adjacent the P2 primer, the following steps can be performed in addition to other routine library creation steps known to those ordinarily skilled in the art: (1) generate DNA fragments by shearing a DNA sample and repairing the ends; (2) ligate LMP CAP adaptors to the ends of the fragmented DNA; (3) circularize the DNA with an internal adaptor which leaves nicks; (4) conduct a nick translation reaction to move the position of the nicks to a new position that is within the DNA fragment (the timing of the nick translation reaction can be stopped to place the nick at any desired position along the DNA fragment); (5) digest the nick translated DNA with T7 exonuclease and S1 nuclease to release the linear, double-stranded mate pair tags; and (6) ligate multiplex P1 and P2 barcoded adaptors to the mate pair tags.

In some embodiments, the amplified library can be quantitated by qPCR or other method. In some embodiments, the libraries can be pooled. In some embodiments, beads can be templated with the mate pair library by emulsion PCR. The templated beads can be sequenced. In the mate pair library, the P1 and IA end of the insert sequences can be sequenced, and the barcode can be sequenced, in three separate reads from the same strand.

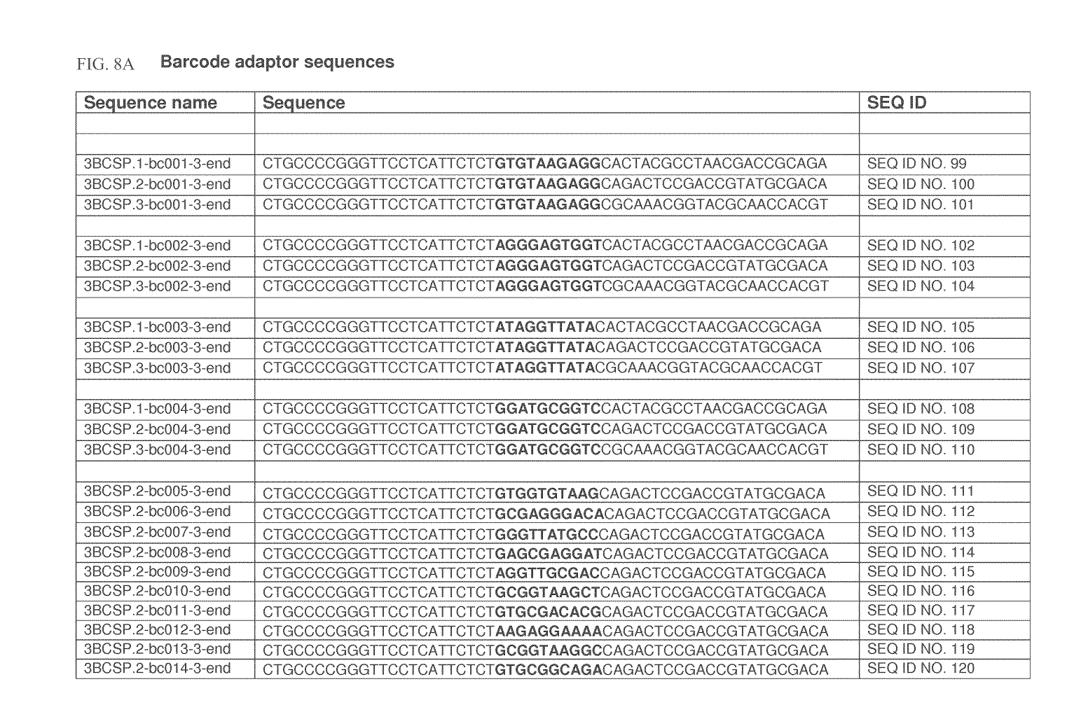

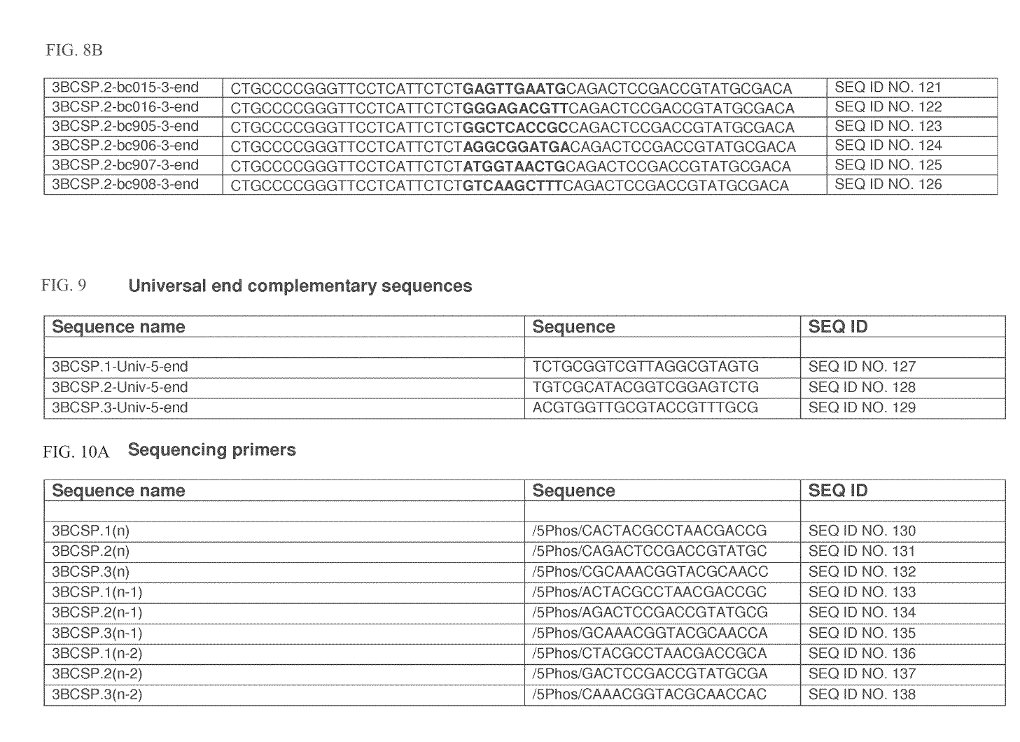

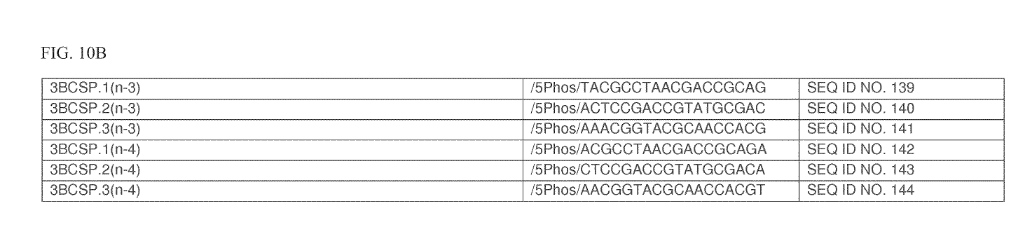

The barcode can be sequenced using barcode adaptor sequences having P2, barcode, and priming sequences, such as those shown in FIGS. 8A and B (SEQ ID NOS:99-126), shown as reverse complements with the barcode sequences in bold. Examples of Universal end complementary sequences are shown in FIG. 9 (SEQ ID NOS:127-129). Examples of sequencing primers are shown in FIG. 10 (SEQ ID NOS:130-138).

### Paired End Libraries

The nucleic acid barcodes can be adapted for use in generating paired end libraries. Generally, the paired end libraries can be constructed by: fragmenting a starting source of DNA (e.g., shearing); and attaching P1 adaptors and barcoded P2 adaptors to the ends of the fragments. The paired end library can be amplified and sequenced. In the paired end library, the paired ends and the barcodes can be sequenced in separate reads from the same strand.

### SAGE Libraries

The nucleic acid barcodes described above can be adapted to construct a nucleic acid library for use in gene expression analysis using nucleic acid sequencing. For example, the nucleic acid barcodes can be used in SOLiD™ SAGE™ gene expression analysis (where SAGE™ is Serial Analysis of Gene Expression) developed by Applied Biosystems (now Life Technologies, Inc.).

In some embodiments, the barcodes can lack one or more restriction enzyme recognition sequence(s), amplification sequences, or adaptor sequences that are used for constructing the nucleic acid library. For example, in SAGE™, a recognition site for the restriction enzyme EcoP15I is used to generate SAGE™ tags. Therefore, nucleic acid barcodes used in SAGE™, other gene expression analysis, or other analyses reliant on recognition sites for restriction enzymes, etc., can be designed to avoid recognition sites necessary for the further analysis carried out in those processes.

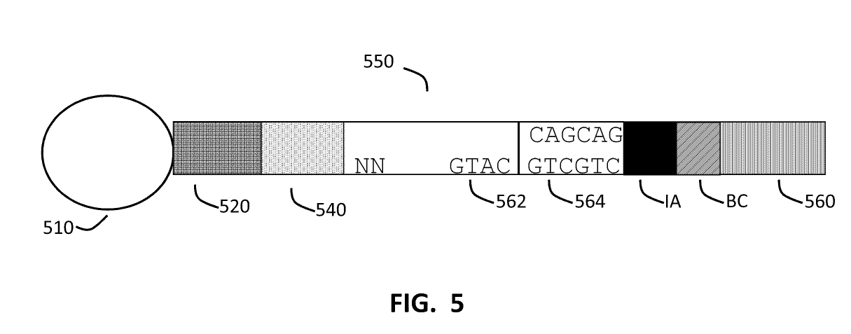

In some embodiments, SAGE™-compatible nucleic acid barcodes can be designed to be positioned adjacent the P1 primer. SAGE™ tags have a 2-base overhang resulting from EcoP15I cleavage. To account for the overhang, the nucleic acid barcode can comprise an overhang end having 1, 2, 3, 4, 5, or longer overhang end. The overhang end can include a degenerate sequence. The nucleic acid barcode can include a 2-nucleotide degenerate extension to ligate to the SAGE™ tag. Alternatively, the 2-base overhang on the SAGE™ tag can be degraded or filled-in to produce a blunt end for ligating to the nucleic acid barcode. FIG. 4A schematically depicts a nucleic acid barcode BC attached to a P1 primer 440, wherein the nucleic acid barcode BC comprises a 2-nucleotide degenerate extension NN.

The P2 primer can be adapted to ligate properly to the SAGE™ tag. The P2 primer can have an NIaIII overhang (GTAC) attached to an EcoP15I recognition site to ligate to the SAGE™ tag. FIG. 4B schematically depicts a SAGE™ tag 450 ligated to nucleic acid barcode BC and the NIaIII overhang 462 and EcoP15I recognition site 464, which are ligated to P2 primer 460. P1 primer 440 is attached to solid support 410 (e.g., bead) through linker 420.

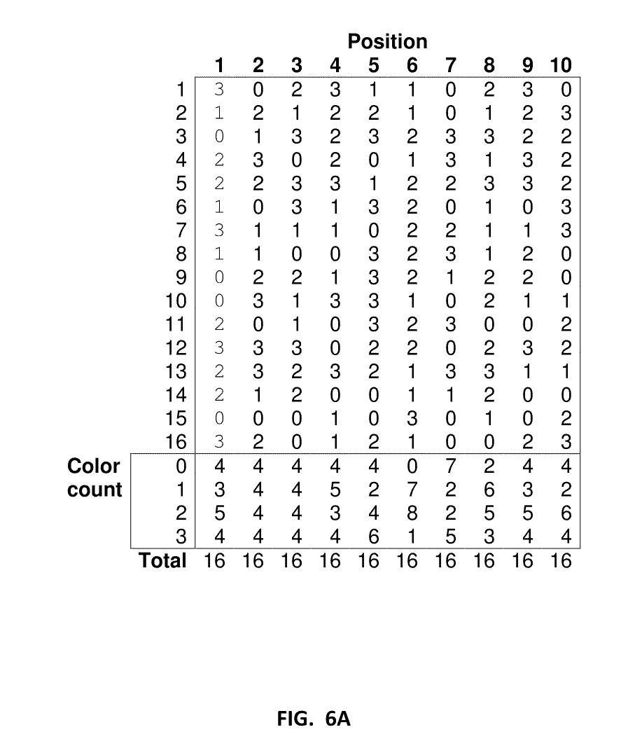

In some embodiments, the nucleic acid barcode can be positioned adjacent the P2 primer for SAGE™ analysis. In embodiments where the nucleic acid barcode is positioned adjacent the P2 primer, a barcoding adaptor can be used to connect the SAGE™ tag to the nucleic acid barcode. The barcoding adaptor can also include an internal adaptor, which can be similar to the internal adaptor 356 described above with respect to FIG. 3, with a NIaIII overhang to ligate to the SAGE™ tag and an EcoP15I recognition site. The P1 primer can also comprise a 2-nucleotide degenerate overhang to ligate to the SAGE™ tag. FIG. 5 schematically depicts nucleic acid barcode BC positioned adjacent a P2 primer 560. Primer P1 540 is attached to a solid support 510 (e.g., a bead) through linker 520. A 2-nucleotide degenerate overhang NN allows a SAGE™ tag 550 to ligate to the P1 primer 540. On the other side of the SAGE™ tag 550, an internal adapter IA is ligated to an EcoP15I recognition site 564 and an NIaIII overhang 562. In accordance with at least one embodiment of the present teachings, the nucleic acid barcode can be incorporated in an oligonucleotide comprising one or more oligonucleotide sequences, such as, for example, an internal adapter and a P2 primer. For example, in at least one embodiment, the nucleic acid barcode can be incorporated in an oligonucleotide comprising a modified internal adapter, the nucleic acid barcode, and a P2 primer. In some embodiments, the barcode need not be part of the library construct, but can be introduced by PCR amplification using a primer having the barcode sequence.

To generate barcoded SAGE™ libraries using nucleic acid barcodes positioned adjacent the P2 primer, the following steps can be performed in addition to other routine library creation steps known to those ordinarily skilled in the art: (1) generate an immobilized cDNA library from poly-A RNA; (2) digest the cDNA with a restriction enzyme to create cohesive ends for EcoP151 ends (e.g., digest with NIa III); (3) ligate to the NIa III cut ends an internal adaptor having cohesive ends for EcoP151 to form an EcoP151 recognition site; (4) cleave the EcoP15I site to generate SAGE™ tag fragments; (5) ligate P1 adaptors (e.g., SAGE™-specific P1 adaptors have a 2-base degenerate extension to hybridize with the overhang from the cleaved EcoP15I ends); and (6) amplify the library (e.g., PCR using primers having a P2 adaptor and barcode sequences).

In some embodiments, the PCR primers used in step 6 can include the general sequence:

(SEQ ID NO: 140)
5′-CTGCCCCGGGTTCCTCATTCTCTNNNNNNNNNNCTGCTGTACGGCCAAGGCG-3′
P2 sequence barcode Internal Adaptor(IA)

In some embodiments, the amplified library can be quantitated by qPCR or other method. In some embodiments, the libraries can be pooled. In some embodiments, beads can be templated with the library by emulsion PCR. The templated beads can be sequenced.

### Yeast Barcode Libraries

In some embodiments, the nucleic acid barcodes can be used in combination with conventional yeast barcodes, such as those described, for example, by Yan et al., “Yeast Barcoders: a chemogenomic application of a universal donor-strain collection carrying bar-code identifiers,” Nature Methods, 5, pp. 719-725 (2008). Yeast barcodes are unique sequences identifying about 6,000 Saccharomyces cerevisiae gene deletion strains. Conventional yeast barcodes comprise a signature sequence of about 20 bases that are flanked by conserved PCR primer sequences. In at least one embodiment, a set of nucleic acid barcodes comprising about 100 uniquely identifiable barcodes can be used with the 6,000 yeast barcodes, resulting in about 600,000 targets to be analyzed per location (e.g., per location on a slide when using a SOLiD™ sequencing platform). In one further example, a SOLiD™ slide can comprise 8 individual sections, which would provide capacity for about 4.8 million targets. When using both slides in a SOLiD™ apparatus, about 9.6 million targets could be analyzed simultaneously.

In some embodiments, a set of nucleic acid barcodes can be combined with at least one yeast barcode to prepare a module to be analyzed. The module can comprise a first conserved PCR primer adjacent the P1 primer. The nucleic acid barcode can be ligated to the P2 primer between the P2 primer and a second conserved PCR primer. An internal adapter can be positioned between the nucleic acid barcode and the second conserved PCR primer. In at least one embodiment, the complete nucleic acid sequence can comprise a P1 primer, a first conserved PCR primer, an insert with a yeast barcode, a second conserved PCR primer, an internal adapter, a nucleic acid barcode, and a P2 primer.

In at least one embodiment, the first conserved PCR primer comprises the sequence 5′-GATGTCCACGATGGTCTCT-3′ (SEQ ID NO. 97) and the second conserved PCR primer comprises the sequence 5′-GTCGACCTGCAGCGTACG-3′ (SEQ ID NO. 98).

In at least one embodiment, a sequencing experiment is performed wherein one or more chemical compounds are tested against each of the 6,000 Saccharomyces cerevisiae gene deletion strains. Each chemical compound is identified by a uniquely identifiable nucleic acid barcode. Each of the 6,000 Saccharomyces cerevisiae gene deletion strains is identified by a uniquely identifiable yeast barcode.

### ChIP-Seq Libraries

In some embodiments, the nucleic acid barcodes can be adapted for use in generating ChIP-based libraries for nucleic acid sequencing. Chromatin immunoprecipitation (ChIP) technologies involve isolating genomic nucleic acids that are associated with DNA-binding proteins. The chromatin/protein complexes can be isolated using a SOLiD™ ChIP-Seq Kit from Applied Biosystems (now part of Life Technologies). The isolated chromatin/protein complexes can be manipulated and ligated to nucleic acid barcodes and barcodes adaptors to construct a ChIP-based library.

The general steps for chromatin immunoprecipitation can include: (1) treat live cells or tissue with formaldehyde to crosslink proximal molecules to create protein/DNA complexes; (2) lyse the cells to release the cross-linked complexes; (3) fragment the DNA (e.g., via sonication); (4) immunoprecipitate the protein/DNA complex of interest using certain antibodies conjugated to beads; (5) release the DNA from the cross-linked complex by heat treatment; (6) purify the released DNA.

The general steps for preparing the ChIP-based library include: (1) generating cohesive ends on the ChIP-isolated DNA (e.g., end-repair); and (2) attaching P1, P2 and/or barcoded adaptors to the ends of the ChIP-isolated DNA. Nick translation can be performed on the adaptor-ligated DNA to close any gaps or nicks between the DNA fragment and the adaptors. In some embodiments, the ChIP-based library includes fragments of chromatin ligated at the ends with any combination of P1, P2, and/or barcoded adaptors.

### SOLID™ Sequencing System

The libraries having barcodes or barcoded adaptors can be sequenced using any nucleic acid sequencing technology, including the SOLiD™ sequencing system (WO 2006/084132). The SOLiD™ sequencing system includes performing successive cycles of duplex extension along a single-stranded template (FIG. 11, top row). In general, the cycles comprise the steps of extension and ligation. Extension can start from a duplex formed by an initializing oligonucleotide annealed to the template. The initializing oligonucleotide is extended by hybridizing an oligonucleotide probe (e.g., fluorophore-encoded dibase probe) to the template at a position that is adjacent to the initializing oligonucleotide, and ligating the oligonucleotide probe to the initializing oligonucleotide thereby forming an extended duplex. The initializing oligonucleotide is repeatedly extended by successive cycles of hybridization and ligation. The oligonucleotide probe can be labeled, for example, with a fluorophore. The oligonucleotide probe is a member of a family of probes. The label corresponds to the probe family to which the probe belongs. Detection of the fluorophore identifies the family to which to probe belongs (color calling) but does not identify any individual single nucleotide in the oligonucleotide probe during each hybridization-ligation cycle.

Successive cycles of hybridization, ligation, and detection produces an ordered list of probe families to which successive ligated probes belong. The ordered list of probe families is used to obtain information about the sequence. However, knowing to which probe family a newly ligated probe belongs is not by itself sufficient to determine the identity of a nucleotide in the template. Instead, knowing to which probe family the newly ligated probe belongs eliminates certain sequences as possibilities for the sequence of the probe but leaves at least two possibilities for the identity of the nucleotide at each position.

In some embodiments, after performing a desired number of cycles, a first set of candidate sequences is generated using the ordered series of probe family identities. The first set of candidate sequences may provide sufficient information to determine the sequence of the template. In some embodiments, after several cycles of successive ligation reactions, the extended duplex can be removed from the template, and another round of successive cycles of hybridization, ligation, and detection can be performed, using an initializing oligonucleotide that hybridizes to the template at a position that is off-set by one base (FIG. 11, second, third, fourth, and fifth rows).

### SOLiD™ Color Calling

In some embodiments, each oligonucleotide probe assays two or more base positions (e.g., overlapping dibase color positions) in the template at a time. In some embodiments, the SOLiD™ sequencing system can use four more different fluorescent dyes to encode for the sixteen possible two-base combinations (dibase color calling). The sequence of the template is represented as an initial base followed by a sequence of overlapping dimers (adjacent pairs of bases). The system encodes each dimer with one of four colors using a degenerate coding scheme that satisfies a number of rules. A single color in the read can represent any of four dimers, but the overlapping properties of the dimers and the nature of the color code allow for error-correcting properties. The SOLiD System's 2 base color coding scheme is shown Table 1.

For example, the DNA sequence 5′-ATCAAGCCTC-3′ (SEQ ID NO:141) can be color encoded by the steps of: (1) the di-base AT is encoded by “3” as shown in Table 1; (2) advance the DNA sequence by one base and the di-base TC di-base is encoded by “2” as shown in Table 1; (3) continue color encoding the remainder of the template to yield the color position shown below.

| Base Sequence: | A T C A A G C C T C | (SEQ ID NO: 142) |
|---|---|---|
| Color code: | 3 2 1 0 2 3 0 2 2 | |

Although various embodiments are described with reference SOLiD™ and di-base sequencing techniques, it should be understood that the nucleic acid barcode principles can be applied to other next generation sequencing techniques and in particular can be useful with next generation multiplex sequencing. The nucleic acid barcodes according to the present teachings can be adapted for other applications requiring the unique identification of nucleic acid samples. Those ordinarily skilled in the art would understand how to make modifications to the lengths, design, sequences, etc. of the nucleic acid barcodes to optimize applicability in other sequencing systems/techniques, as well as other applications requiring the unique identification of nucleic acid samples.

In some embodiments, identifier codes, such as proteins, can be sequenced using well known methods, including Edman degradation (Edman 1950 Acta Chem Scand. 4:283-293; and NiaII 1973 Meth. Enzymol. 27:942-1010)) or mass spectrometry (Hernandez 2006 Mass Spectrometry Reviews 25:235-254; Snijders 2005 Journal Proteome Res. 4:578-585; Miyagi 2007 Mass Spectrometry Reviews 26:121-136; and Haqqani 2008 Methods Mol. Biol. 439:241-256).

While the principles of the present teachings have been described in connection with specific embodiments of nucleic acid barcodes and sequencing platforms, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

1. A composition comprising a plurality of identifier codes
a) each identifier code being comprised of a sequence of from 4 to 30 individual subunits;
b) the sequence of subunits of each identifier code being distinguishable from the sequence of subunits of each other member of the plurality of identifier codes;
c) wherein the sequence of subunits of each identifier code:
(i) lacks any contiguous sequence of four or more identical subunits; and
(ii) differs by at least three subunits from the sequence of subunits of each other member of the plurality of identifier codes.

2. A composition comprising a plurality of identifier codes
a) each identifier code being comprised of a sequence of from 4 to 30 individual subunits;
b) wherein a detectable signal is associated with each subunit or with pairs or sets of subunits such that each identifier code has a sequence of detectable signals associated with it;
c) each sequence of detectable signals being distinguishable from the sequence of detectable signals of each other member of the plurality of identifier codes;
d) wherein the sequence of detectable signals of each identifier code:
(iii) lacks any contiguous sequence of four or more identical detectable signals; and
(iv) differs by at least three detectable signals from the sequence of subunits of each other member of the plurality of identifier codes.

3. A system comprising a plurality of individually identifiable nucleic acid barcodes comprising overlapping dibase color positions which are sequenced in a color space with at least two fluorophore encoded dibase probes in a fluorophore color calling dibase sequencing system, wherein the plurality of nucleic acid barcodes are designed to yield a color call that lacks repeating one fluorophore color that is called 4 or more times in a row.

4. A system comprising a plurality of individually identifiable nucleic acid barcodes comprising overlapping dibase color positions which are sequenced in a color space with at least two fluorophore encoded dibase probes in a fluorophore color calling dibase sequencing system, wherein the plurality of nucleic acid barcodes are designed to yield a color balance having the colors of the at least two fluorophore encoded dibase probes called at least once in all color positions of the barcode.

5. A system comprising a plurality of individually identifiable nucleic acid barcodes comprising overlapping dibase color positions which are sequenced in a color space with at least two fluorophore encoded dibase probes in a fluorophore color calling dibase sequencing system, wherein the plurality of nucleic acid barcodes are designed to yield a color call of any two nucleic acid barcodes will differ in at least three of the same color positions of both barcodes.

6. A system comprising a plurality of individually identifiable nucleic acid barcodes comprising overlapping dibase color positions which are sequenced in a color space with at least two fluorophore encoded dibase probes in a fluorophore color calling dibase sequencing system, wherein the plurality of nucleic acid barcodes are designed to yield a nested subset which satisfies the criterion that the plurality of the nucleic acid barcodes satisfies.

7. The system of claim 6, wherein the plurality of nucleic acid barcodes are designed to be an ordered list of nested barcodes comprising at least two barcodes having a different color call in 3 positions of the first 3 color positions of the at least two barcodes.

8. The system of claim 6, wherein the plurality of nucleic acid barcodes are designed to be an ordered list of nested barcodes comprising at least two barcodes having a different color call in 3 positions of the first 4 color positions of the at least two barcodes.

9. The system of claim 6, wherein the plurality of nucleic acid barcodes are designed to be an ordered list of nested barcodes comprising at least two barcodes having a different color call in 3 positions of the first 5 color positions of the at least two barcodes.

10. The system of claim 3, wherein the individually identifiable nucleic acid barcodes are 4-30 bases in length.

11. The system of claim 3, wherein the individually identifiable nucleic acid barcodes are ligated to a first nucleic acid priming site (P1).

12. The system of claim 3, wherein the individually identifiable nucleic acid barcodes are ligated to a second nucleic acid priming site (P2).

13. The system of claim 3, wherein the individually identifiable nucleic acid barcodes are ligated to a nucleic acid internal adaptor (IA).

14. The system of claim 3, wherein the individually identifiable nucleic acid barcodes are ligated between a first nucleic acid priming site and a nucleic acid internal adaptor (IA), or between a nucleic acid internal adaptor (IA) and a second nucleic acid priming site (P2).

15. The system of claim 3, wherein the individually identifiable nucleic acid barcodes comprises a restriction endonuclease recognition sequence.

16. The system of claim 15, wherein the restriction endonuclease recognition sequence is EcoP151.

17. The system of claim 3, wherein the individually identifiable nucleic acid barcodes comprises an overhang sequence.

18. The system of claim 17, wherein the overhang sequence is compatible with a restriction endonuclease recognition sequence.

19. The system of claim 3, comprising individually identifiable nucleic acid barcodes selected from a group consisting of SEQ ID NOS:1-96.

20. The system of claim 3, comprising individually identifiable nucleic acid barcodes selected from a group consisting of SEQ ID NOS:1-4; SEQ ID NOS:5-8; SEQ ID NOS:9-12; SEQ ID NOS:13-16; SEQ ID NOS:17-20; SEQ ID NOS:21-24; SEQ ID NOS:25-28; SEQ ID NOS:29-32; SEQ ID NOS:33-36; SEQ ID NOS:37-40; SEQ ID NOS:41-44; SEQ ID NOS:45-48; SEQ ID NOS:49-52; SEQ ID NOS:53-56; SEQ ID NOS:57-60; SEQ ID NOS:61-64; SEQ ID NOS:65-68; SEQ ID NOS:69-72; SEQ ID NOS:73-76; SEQ ID NOS:77-80; SEQ ID NOS:81-84; SEQ ID NOS:85-88; SEQ ID NOS:89-92; and SEQ ID NOS:93-96.

21. A multiplex nucleic acid library comprising a plurality of sample nucleic acids attached to the plurality of individually identifiable nucleic acid barcodes of claim 3.

22. The multiplex nucleic acid library of claim 21 attached to a solid surface.

23. A method for identifying multiplexed samples, comprising:
a) attaching a plurality of sample nucleic acids to a plurality of individually identifiable nucleic acid barcodes of claim 3; and
b) sequencing the plurality of sample nucleic acids and the plurality of individually identifiable nucleic acid barcodes.

24. A composition comprising an individually identifiable nucleic acid barcode comprising overlapping dibase color positions which are sequenced in a color space with at least two fluorophore encoded dibase probes in a fluorophore color calling dibase sequencing system, wherein the nucleic acid barcode is designed to yield a color call that lacks repeating one fluorophore color that is called 4 or more times in a row.

25. A composition comprising an individually identifiable nucleic acid barcodes comprising overlapping dibase color positions which are sequenced in a color space with at least two fluorophore encoded dibase probes in a fluorophore color calling dibase sequencing system, wherein the nucleic acid barcode is designed to yield a color balance having the colors of the at least two fluorophore encoded dibase probes called at least once in all color positions of the barcode.

26. A composition comprising an individually identifiable nucleic acid barcode comprising overlapping dibase color positions which are sequenced in a color space with at least two fluorophore encoded dibase probes in a fluorophore color calling dibase sequencing system, wherein the nucleic acid barcode is designed to yield a color call of any two nucleic acid barcodes that differ in at least three of the same color positions of both barcodes.

27. The composition of claim 24, wherein the individually identifiable nucleic acid barcodes are 4-30 bases in length.

28. The composition of claim 24, wherein the individually identifiable nucleic acid barcodes are ligated to a first nucleic acid priming site (P1).

29. The composition of claim 24, wherein the individually identifiable nucleic acid barcodes are ligated to a second nucleic acid priming site (P2).

30. The composition of claim 24, wherein the individually identifiable nucleic acid barcodes are ligated to a nucleic acid internal adaptor (IA).

31. The composition of claim 24, wherein the individually identifiable nucleic acid barcodes are ligated between a first nucleic acid priming site and a nucleic acid internal adaptor (IA), or between a nucleic acid internal adaptor (IA) and a second nucleic acid priming site (P2).

32. The composition of claim 24, wherein the individually identifiable nucleic acid barcodes comprises a restriction endonuclease recognition sequence.

33. The composition of claim 32, wherein the restriction endonuclease recognition sequence is EcoP151.

34. The composition of claim 24, wherein the individually identifiable nucleic acid barcodes comprises an overhang sequence.

35. The composition of claim 24, wherein the overhang sequence is compatible with a restriction endonuclease recognition sequence.

36. A composition comprising any one individually identifiable nucleic acid barcode selected from a group consisting of SEQ ID NOS:1-96.

37. A composition comprising a set of individually identifiable nucleic acid barcodes selected from a group consisting of SEQ ID NOS:1-4; SEQ ID NOS:5-8; SEQ ID NOS:9-12; SEQ ID NOS:13-16; SEQ ID NOS:17-20; SEQ ID NOS:21-24; SEQ ID NOS:25-28; SEQ ID NOS:29-32; SEQ ID NOS:33-36; SEQ ID NOS:37-40; SEQ ID NOS:41-44; SEQ ID NOS:45-48; SEQ ID NOS:49-52; SEQ ID NOS:53-56; SEQ ID NOS:57-60; SEQ ID NOS:61-64; SEQ ID NOS:65-68; SEQ ID NOS:69-72; SEQ ID NOS:73-76; SEQ ID NOS:77-80; SEQ ID NOS:81-84; SEQ ID NOS:85-88; SEQ ID NOS:89-92; and SEQ ID NOS:93-96.

38. A composition comprising a color position equivalent of any one individually identifiable nucleic acid barcodes selected from a group consisting of SEQ ID NOS:1-96.

39. A composition comprising a set of color position equivalent of individually identifiable nucleic acid barcodes selected from a group consisting of SEQ ID NOS:1-4; SEQ ID NOS:5-8; SEQ ID NOS:9-12; SEQ ID NOS:13-16; SEQ ID NOS:17-20; SEQ ID NOS:21-24; SEQ ID NOS:25-28; SEQ ID NOS:29-32; SEQ ID NOS:33-36; SEQ ID NOS:37-40; SEQ ID NOS:41-44; SEQ ID NOS:45-48; SEQ ID NOS:49-52; SEQ ID NOS:53-56; SEQ ID NOS:57-60; SEQ ID NOS:61-64; SEQ ID NOS:65-68; SEQ ID NOS:69-72; SEQ ID NOS:73-76; SEQ ID NOS:77-80; SEQ ID NOS:81-84; SEQ ID NOS:85-88; SEQ ID NOS:89-92; and SEQ ID NOS:93-96.


SEQUENCE LISTING (149 sequences)

SEQ ID NO: 1 - DNA, 10 bp, Artificial Sequence
  Description of Artificial Sequence Synthetic oligonucleotide
cctcttacac

SEQ ID NO: 2 - DNA, 10 bp, Artificial Sequence
  Description of Artificial Sequence Synthetic oligonucleotide
accactccct

SEQ ID NO: 3 - DNA, 10 bp, Artificial Sequence
  Description of Artificial Sequence Synthetic oligonucleotide
tataacctat

SEQ ID NO: 4 - DNA, 10 bp, Artificial Sequence
  Description of Artificial Sequence Synthetic oligonucleotide
gaccgcatcc

SEQ ID NO: 5 - DNA, 10 bp, Artificial Sequence
  Description of Artificial Sequence Synthetic oligonucleotide
cttacaccac

SEQ ID NO: 6 - DNA, 10 bp, Artificial Sequence
  Description of Artificial Sequence Synthetic oligonucleotide
tgtccctcgc

SEQ ID NO: 7 - DNA, 10 bp, Artificial Sequence
  Description of Artificial Sequence Synthetic oligonucleotide
ggcataaccc

SEQ ID NO: 8 - DNA, 10 bp, Artificial Sequence
  Description of Artificial Sequence Synthetic oligonucleotide
atcctcgctc

SEQ ID NO: 9 - DNA, 10 bp, Artificial Sequence
  Description of Artificial Sequence Synthetic oligonucleotide
gtcgcaacct

SEQ ID NO: 10 - DNA, 10 bp, Artificial Sequence
  Description of Artificial Sequence Synthetic oligonucleotide
agcttaccgc

SEQ ID NO: 11 - DNA, 10 bp, Artificial Sequence
  Description of Artificial Sequence Synthetic oligonucleotide
cgtgtcgcac

SEQ ID NO: 12 - DNA, 10 bp, Artificial Sequence
  Description of Artificial Sequence Synthetic oligonucleotide
ttttcctctt

SEQ ID NO: 13 - DNA, 10 bp, Artificial Sequence
  Description of Artificial Sequence Synthetic oligonucleotide
gccttaccgc

SEQ ID NO: 14 - DNA, 10 bp, Artificial Sequence
  Description of Artificial Sequence Synthetic oligonucleotide
tctgccgcac

SEQ ID NO: 15 - DNA, 10 bp, Artificial Sequence
  Description of Artificial Sequence Synthetic oligonucleotide
cattcaactc

SEQ ID NO: 16 - DNA, 10 bp, Artificial Sequence
  Description of Artificial Sequence Synthetic oligonucleotide
aacgtctccc

SEQ ID NO: 17 - DNA, 10 bp, Artificial Sequence
  Description of Artificial Sequence Synthetic oligonucleotide
gcggtgagcc

SEQ ID NO: 18 - DNA, 10 bp, Artificial Sequence
  Description of Artificial Sequence Synthetic oligonucleotide
tcatccgcct

SEQ ID NO: 19 - DNA, 10 bp, Artificial Sequence
  Description of Artificial Sequence Synthetic oligonucleotide
cagttaccat

SEQ ID NO: 20 - DNA, 10 bp, Artificial Sequence
  Description of Artificial Sequence Synthetic oligonucleotide
aaagcttgac

SEQ ID NO: 21 - DNA, 10 bp, Artificial Sequence
  Description of Artificial Sequence Synthetic oligonucleotide
ggaaccgcac

SEQ ID NO: 22 - DNA, 10 bp, Artificial Sequence
  Description of Artificial Sequence Synthetic oligonucleotide
tcatcttctc

SEQ ID NO: 23 - DNA, 10 bp, Artificial Sequence
  Description of Artificial Sequence Synthetic oligonucleotide
caagcaccgc

SEQ ID NO: 24 - DNA, 10 bp, Artificial Sequence
  Description of Artificial Sequence Synthetic oligonucleotide
ataccgaccc

SEQ ID NO: 25 - DNA, 10 bp, Artificial Sequence
  Description of Artificial Sequence Synthetic oligonucleotide
tcatcatgtt

SEQ ID NO: 26 - DNA, 10 bp, Artificial Sequence
  Description of Artificial Sequence Synthetic oligonucleotide
cgggctcccg

SEQ ID NO: 27 - DNA, 10 bp, Artificial Sequence
  Description of Artificial Sequence Synthetic oligonucleotide
aagtttgctg

SEQ ID NO: 28 - DNA, 10 bp, Artificial Sequence
  Description of Artificial Sequence Synthetic oligonucleotide
gtagtaagct

SEQ ID NO: 29 - DNA, 10 bp, Artificial Sequence
  Description of Artificial Sequence Synthetic oligonucleotide
ccctagattc

SEQ ID NO: 30 - DNA, 10 bp, Artificial Sequence
  Description of Artificial Sequence Synthetic oligonucleotide
tcttcgctac

SEQ ID NO: 31 - DNA, 10 bp, Artificial Sequence
  Description of Artificial Sequence Synthetic oligonucleotide
acgcaccagc

SEQ ID NO: 32 - DNA, 10 bp, Artificial Sequence
  Description of Artificial Sequence Synthetic oligonucleotide
gcacccaacc

SEQ ID NO: 33 - DNA, 10 bp, Artificial Sequence
  Description of Artificial Sequence Synthetic oligonucleotide
gtatccaacg

SEQ ID NO: 34 - DNA, 10 bp, Artificial Sequence
  Description of Artificial Sequence Synthetic oligonucleotide
cctttaacga

SEQ ID NO: 35 - DNA, 10 bp, Artificial Sequence
  Description of Artificial Sequence Synthetic oligonucleotide
tcctacgctt

SEQ ID NO: 36 - DNA, 10 bp, Artificial Sequence
  Description of Artificial Sequence Synthetic oligonucleotide
atgtgagaac

SEQ ID NO: 37 - DNA, 10 bp, Artificial Sequence
  Description of Artificial Sequence Synthetic oligonucleotide
ggtataacag

SEQ ID NO: 38 - DNA, 10 bp, Artificial Sequence
  Description of Artificial Sequence Synthetic oligonucleotide
ctaagacgac

SEQ ID NO: 39 - DNA, 10 bp, Artificial Sequence
  Description of Artificial Sequence Synthetic oligonucleotide
actcacgata

SEQ ID NO: 40 - DNA, 10 bp, Artificial Sequence
  Description of Artificial Sequence Synthetic oligonucleotide
taaccctttt

SEQ ID NO: 41 - DNA, 10 bp, Artificial Sequence
  Description of Artificial Sequence Synthetic oligonucleotide
caatcccaca

SEQ ID NO: 42 - DNA, 10 bp, Artificial Sequence
  Description of Artificial Sequence Synthetic oligonucleotide
tagtacattc

SEQ ID NO: 43 - DNA, 10 bp, Artificial Sequence
  Description of Artificial Sequence Synthetic oligonucleotide
aaccctagcg

SEQ ID NO: 44 - DNA, 10 bp, Artificial Sequence
  Description of Artificial Sequence Synthetic oligonucleotide
gatcatcctt

SEQ ID NO: 45 - DNA, 10 bp, Artificial Sequence
  Description of Artificial Sequence Synthetic oligonucleotide
agccaagtac

SEQ ID NO: 46 - DNA, 10 bp, Artificial Sequence
  Description of Artificial Sequence Synthetic oligonucleotide
ttcgacgacc

SEQ ID NO: 47 - DNA, 10 bp, Artificial Sequence
  Description of Artificial Sequence Synthetic oligonucleotide
gccatccctc

SEQ ID NO: 48 - DNA, 10 bp, Artificial Sequence
  Description of Artificial Sequence Synthetic oligonucleotide
cacttacggc

SEQ ID NO: 49 - DNA, 10 bp, Artificial Sequence
  Description of Artificial Sequence Synthetic oligonucleotide
cttatgacat

SEQ ID NO: 50 - DNA, 10 bp, Artificial Sequence
  Description of Artificial Sequence Synthetic oligonucleotide
gcaagccttc

SEQ ID NO: 51 - DNA, 10 bp, Artificial Sequence
  Description of Artificial Sequence Synthetic oligonucleotide
actcctgctt

SEQ ID NO: 52 - DNA, 10 bp, Artificial Sequence
  Description of Artificial Sequence Synthetic oligonucleotide
ttacaattac

SEQ ID NO: 53 - DNA, 10 bp, Artificial Sequence
  Description of Artificial Sequence Synthetic oligonucleotide
acttgatgac

SEQ ID NO: 54 - DNA, 10 bp, Artificial Sequence
  Description of Artificial Sequence Synthetic oligonucleotide
tccgcctttt

SEQ ID NO: 55 - DNA, 10 bp, Artificial Sequence
  Description of Artificial Sequence Synthetic oligonucleotide
cgcttaagct

SEQ ID NO: 56 - DNA, 10 bp, Artificial Sequence
  Description of Artificial Sequence Synthetic oligonucleotide
ggtgacatgc

SEQ ID NO: 57 - DNA, 10 bp, Artificial Sequence
  Description of Artificial Sequence Synthetic oligonucleotide
ttcttactag

SEQ ID NO: 58 - DNA, 10 bp, Artificial Sequence
  Description of Artificial Sequence Synthetic oligonucleotide
cgccacttta

SEQ ID NO: 59 - DNA, 10 bp, Artificial Sequence
  Description of Artificial Sequence Synthetic oligonucleotide
gacattactt

SEQ ID NO: 60 - DNA, 10 bp, Artificial Sequence
  Description of Artificial Sequence Synthetic oligonucleotide
accgaggcac

SEQ ID NO: 61 - DNA, 10 bp, Artificial Sequence
  Description of Artificial Sequence Synthetic oligonucleotide
cgataatctt

SEQ ID NO: 62 - DNA, 10 bp, Artificial Sequence
  Description of Artificial Sequence Synthetic oligonucleotide
accctcacct

SEQ ID NO: 63 - DNA, 10 bp, Artificial Sequence
  Description of Artificial Sequence Synthetic oligonucleotide
tcgaacccgc

SEQ ID NO: 64 - DNA, 10 bp, Artificial Sequence
  Description of Artificial Sequence Synthetic oligonucleotide
ggtgtagcac

SEQ ID NO: 65 - DNA, 10 bp, Artificial Sequence
  Description of Artificial Sequence Synthetic oligonucleotide
gcttgatccc

SEQ ID NO: 66 - DNA, 10 bp, Artificial Sequence
  Description of Artificial Sequence Synthetic oligonucleotide
acattacatc

SEQ ID NO: 67 - DNA, 10 bp, Artificial Sequence
  Description of Artificial Sequence Synthetic oligonucleotide
ccctaaggac

SEQ ID NO: 68 - DNA, 10 bp, Artificial Sequence
  Description of Artificial Sequence Synthetic oligonucleotide
tcgtcaatgc

SEQ ID NO: 69 - DNA, 10 bp, Artificial Sequence
  Description of Artificial Sequence Synthetic oligonucleotide
aaagcatatc

SEQ ID NO: 70 - DNA, 10 bp, Artificial Sequence
  Description of Artificial Sequence Synthetic oligonucleotide
tctgtagggc

SEQ ID NO: 71 - DNA, 10 bp, Artificial Sequence
  Description of Artificial Sequence Synthetic oligonucleotide
cgttccctgt

SEQ ID NO: 72 - DNA, 10 bp, Artificial Sequence
  Description of Artificial Sequence Synthetic oligonucleotide
gtattcactt

SEQ ID NO: 73 - DNA, 10 bp, Artificial Sequence
  Description of Artificial Sequence Synthetic oligonucleotide
acgtcattgc

SEQ ID NO: 74 - DNA, 10 bp, Artificial Sequence
  Description of Artificial Sequence Synthetic oligonucleotide
tcagcgtcct

SEQ ID NO: 75 - DNA, 10 bp, Artificial Sequence
  Description of Artificial Sequence Synthetic oligonucleotide
gcccagatac

SEQ ID NO: 76 - DNA, 10 bp, Artificial Sequence
  Description of Artificial Sequence Synthetic oligonucleotide
cctaaaactt

SEQ ID NO: 77 - DNA, 10 bp, Artificial Sequence
  Description of Artificial Sequence Synthetic oligonucleotide
aagaccagat

SEQ ID NO: 78 - DNA, 10 bp, Artificial Sequence
  Description of Artificial Sequence Synthetic oligonucleotide
gatgattgcc

SEQ ID NO: 79 - DNA, 10 bp, Artificial Sequence
  Description of Artificial Sequence Synthetic oligonucleotide
taattctact

SEQ ID NO: 80 - DNA, 10 bp, Artificial Sequence
  Description of Artificial Sequence Synthetic oligonucleotide
caccgtaaac

SEQ ID NO: 81 - DNA, 10 bp, Artificial Sequence
  Description of Artificial Sequence Synthetic oligonucleotide
aatgacgttc

SEQ ID NO: 82 - DNA, 10 bp, Artificial Sequence
  Description of Artificial Sequence Synthetic oligonucleotide
ctcccttcac

SEQ ID NO: 83 - DNA, 10 bp, Artificial Sequence
  Description of Artificial Sequence Synthetic oligonucleotide
tacgccatcc

SEQ ID NO: 84 - DNA, 10 bp, Artificial Sequence
  Description of Artificial Sequence Synthetic oligonucleotide
gttcatccgc

SEQ ID NO: 85 - DNA, 10 bp, Artificial Sequence
  Description of Artificial Sequence Synthetic oligonucleotide
aacgctttcc

SEQ ID NO: 86 - DNA, 10 bp, Artificial Sequence
  Description of Artificial Sequence Synthetic oligonucleotide
tcctggtact

SEQ ID NO: 87 - DNA, 10 bp, Artificial Sequence
  Description of Artificial Sequence Synthetic oligonucleotide
gctttgctat

SEQ ID NO: 88 - DNA, 10 bp, Artificial Sequence
  Description of Artificial Sequence Synthetic oligonucleotide
catgatcaac

SEQ ID NO: 89 - DNA, 10 bp, Artificial Sequence
  Description of Artificial Sequence Synthetic oligonucleotide
tagacagcct

SEQ ID NO: 90 - DNA, 10 bp, Artificial Sequence
  Description of Artificial Sequence Synthetic oligonucleotide
agtaggtcac

SEQ ID NO: 91 - DNA, 10 bp, Artificial Sequence
  Description of Artificial Sequence Synthetic oligonucleotide
cccaatacgc

SEQ ID NO: 92 - DNA, 10 bp, Artificial Sequence
  Description of Artificial Sequence Synthetic oligonucleotide
gtaatccctt

SEQ ID NO: 93 - DNA, 10 bp, Artificial Sequence
  Description of Artificial Sequence Synthetic oligonucleotide
gcatcgtaac

SEQ ID NO: 94 - DNA, 10 bp, Artificial Sequence
  Description of Artificial Sequence Synthetic oligonucleotide
aaacacccat

SEQ ID NO: 95 - DNA, 10 bp, Artificial Sequence
  Description of Artificial Sequence Synthetic oligonucleotide
tgccggactc

SEQ ID NO: 96 - DNA, 10 bp, Artificial Sequence
  Description of Artificial Sequence Synthetic oligonucleotide
ctcttcgatt

SEQ ID NO: 97 - DNA, 19 bp, Artificial Sequence
  Description of Artificial Sequence Synthetic primer
gatgtccacgatggtctct

SEQ ID NO: 98 - DNA, 18 bp, Artificial Sequence
  Description of Artificial Sequence Synthetic primer
gtcgacctgcagcgtacg

SEQ ID NO: 99 - DNA, 55 bp, Artificial Sequence
  Description of Artificial Sequence Synthetic oligonucleotide
ctgccccgggttcctcattctctgtgtaagaggcactacgcctaacgaccgcaga

SEQ ID NO: 100 - DNA, 55 bp, Artificial Sequence
  Description of Artificial Sequence Synthetic oligonucleotide
ctgccccgggttcctcattctctgtgtaagaggcagactccgaccgtatgcgaca

SEQ ID NO: 101 - DNA, 55 bp, Artificial Sequence
  Description of Artificial Sequence Synthetic oligonucleotide
ctgccccgggttcctcattctctgtgtaagaggcgcaaacggtacgcaaccacgt

SEQ ID NO: 102 - DNA, 55 bp, Artificial Sequence
  Description of Artificial Sequence Synthetic oligonucleotide
ctgccccgggttcctcattctctagggagtggtcactacgcctaacgaccgcaga

SEQ ID NO: 103 - DNA, 55 bp, Artificial Sequence
  Description of Artificial Sequence Synthetic oligonucleotide
ctgccccgggttcctcattctctagggagtggtcagactccgaccgtatgcgaca

SEQ ID NO: 104 - DNA, 55 bp, Artificial Sequence
  Description of Artificial Sequence Synthetic oligonucleotide
ctgccccgggttcctcattctctagggagtggtcgcaaacggtacgcaaccacgt

SEQ ID NO: 105 - DNA, 55 bp, Artificial Sequence
  Description of Artificial Sequence Synthetic oligonucleotide
ctgccccgggttcctcattctctataggttatacactacgcctaacgaccgcaga

SEQ ID NO: 106 - DNA, 55 bp, Artificial Sequence
  Description of Artificial Sequence Synthetic oligonucleotide
ctgccccgggttcctcattctctataggttatacagactccgaccgtatgcgaca

SEQ ID NO: 107 - DNA, 55 bp, Artificial Sequence
  Description of Artificial Sequence Synthetic oligonucleotide
ctgccccgggttcctcattctctataggttatacgcaaacggtacgcaaccacgt

SEQ ID NO: 108 - DNA, 55 bp, Artificial Sequence
  Description of Artificial Sequence Synthetic oligonucleotide
ctgccccgggttcctcattctctggatgcggtccactacgcctaacgaccgcaga

SEQ ID NO: 109 - DNA, 55 bp, Artificial Sequence
  Description of Artificial Sequence Synthetic oligonucleotide
ctgccccgggttcctcattctctggatgcggtccagactccgaccgtatgcgaca

SEQ ID NO: 110 - DNA, 55 bp, Artificial Sequence
  Description of Artificial Sequence Synthetic oligonucleotide
ctgccccgggttcctcattctctggatgcggtccgcaaacggtacgcaaccacgt

SEQ ID NO: 111 - DNA, 55 bp, Artificial Sequence
  Description of Artificial Sequence Synthetic oligonucleotide
ctgccccgggttcctcattctctgtggtgtaagcagactccgaccgtatgcgaca

SEQ ID NO: 112 - DNA, 55 bp, Artificial Sequence
  Description of Artificial Sequence Synthetic oligonucleotide
ctgccccgggttcctcattctctgcgagggacacagactccgaccgtatgcgaca

SEQ ID NO: 113 - DNA, 55 bp, Artificial Sequence
  Description of Artificial Sequence Synthetic oligonucleotide
ctgccccgggttcctcattctctgggttatgcccagactccgaccgtatgcgaca

SEQ ID NO: 114 - DNA, 55 bp, Artificial Sequence
  Description of Artificial Sequence Synthetic oligonucleotide
ctgccccgggttcctcattctctgagcgaggatcagactccgaccgtatgcgaca

SEQ ID NO: 115 - DNA, 55 bp, Artificial Sequence
  Description of Artificial Sequence Synthetic oligonucleotide
ctgccccgggttcctcattctctaggttgcgaccagactccgaccgtatgcgaca

SEQ ID NO: 116 - DNA, 55 bp, Artificial Sequence
  Description of Artificial Sequence Synthetic oligonucleotide
ctgccccgggttcctcattctctgcggtaagctcagactccgaccgtatgcgaca

SEQ ID NO: 117 - DNA, 55 bp, Artificial Sequence
  Description of Artificial Sequence Synthetic oligonucleotide
ctgccccgggttcctcattctctgtgcgacacgcagactccgaccgtatgcgaca

SEQ ID NO: 118 - DNA, 55 bp, Artificial Sequence
  Description of Artificial Sequence Synthetic oligonucleotide
ctgccccgggttcctcattctctaagaggaaaacagactccgaccgtatgcgaca

SEQ ID NO: 119 - DNA, 55 bp, Artificial Sequence
  Description of Artificial Sequence Synthetic oligonucleotide
ctgccccgggttcctcattctctgcggtaaggccagactccgaccgtatgcgaca

SEQ ID NO: 120 - DNA, 55 bp, Artificial Sequence
  Description of Artificial Sequence Synthetic oligonucleotide
ctgccccgggttcctcattctctgtgcggcagacagactccgaccgtatgcgaca

SEQ ID NO: 121 - DNA, 55 bp, Artificial Sequence
  Description of Artificial Sequence Synthetic oligonucleotide
ctgccccgggttcctcattctctgagttgaatgcagactccgaccgtatgcgaca

SEQ ID NO: 122 - DNA, 55 bp, Artificial Sequence
  Description of Artificial Sequence Synthetic oligonucleotide
ctgccccgggttcctcattctctgggagacgttcagactccgaccgtatgcgaca

SEQ ID NO: 123 - DNA, 55 bp, Artificial Sequence
  Description of Artificial Sequence Synthetic oligonucleotide
ctgccccgggttcctcattctctggctcaccgccagactccgaccgtatgcgaca

SEQ ID NO: 124 - DNA, 55 bp, Artificial Sequence
  Description of Artificial Sequence Synthetic oligonucleotide
ctgccccgggttcctcattctctaggcggatgacagactccgaccgtatgcgaca

SEQ ID NO: 125 - DNA, 55 bp, Artificial Sequence
  Description of Artificial Sequence Synthetic oligonucleotide
ctgccccgggttcctcattctctatggtaactgcagactccgaccgtatgcgaca

SEQ ID NO: 126 - DNA, 55 bp, Artificial Sequence
  Description of Artificial Sequence Synthetic oligonucleotide
ctgccccgggttcctcattctctgtcaagctttcagactccgaccgtatgcgaca

SEQ ID NO: 127 - DNA, 22 bp, Artificial Sequence
  Description of Artificial Sequence Synthetic oligonucleotide
tctgcggtcgttaggcgtagtg

SEQ ID NO: 128 - DNA, 22 bp, Artificial Sequence
  Description of Artificial Sequence Synthetic oligonucleotide
tgtcgcatacggtcggagtctg

SEQ ID NO: 129 - DNA, 22 bp, Artificial Sequence
  Description of Artificial Sequence Synthetic oligonucleotide
acgtggttgcgtaccgtttgcg

SEQ ID NO: 130 - DNA, 18 bp, Artificial Sequence
  Description of Artificial Sequence Synthetic primer
cactacgcctaacgaccg

SEQ ID NO: 131 - DNA, 18 bp, Artificial Sequence
  Description of Artificial Sequence Synthetic primer
cagactccgaccgtatgc

SEQ ID NO: 132 - DNA, 18 bp, Artificial Sequence
  Description of Artificial Sequence Synthetic primer
cgcaaacggtacgcaacc

SEQ ID NO: 133 - DNA, 18 bp, Artificial Sequence
  Description of Artificial Sequence Synthetic primer
actacgcctaacgaccgc

SEQ ID NO: 134 - DNA, 18 bp, Artificial Sequence
  Description of Artificial Sequence Synthetic primer
agactccgaccgtatgcg

SEQ ID NO: 135 - DNA, 18 bp, Artificial Sequence
  Description of Artificial Sequence Synthetic primer
gcaaacggtacgcaacca

SEQ ID NO: 136 - DNA, 18 bp, Artificial Sequence
  Description of Artificial Sequence Synthetic primer
ctacgcctaacgaccgca

SEQ ID NO: 137 - DNA, 18 bp, Artificial Sequence
  Description of Artificial Sequence Synthetic primer
gactccgaccgtatgcga

SEQ ID NO: 138 - DNA, 18 bp, Artificial Sequence
  Description of Artificial Sequence Synthetic primer
caaacggtacgcaaccac

SEQ ID NO: 139 - DNA, 18 bp, Artificial Sequence
  Description of Artificial Sequence Synthetic primer
tacgcctaacgaccgcag

SEQ ID NO: 140 - DNA, 18 bp, Artificial Sequence
  Description of Artificial Sequence Synthetic primer
actccgaccgtatgcgac

SEQ ID NO: 141 - DNA, 18 bp, Artificial Sequence
  Description of Artificial Sequence Synthetic primer
aaacggtacgcaaccacg

SEQ ID NO: 142 - DNA, 18 bp, Artificial Sequence
  Description of Artificial Sequence Synthetic primer
acgcctaacgaccgcaga

SEQ ID NO: 143 - DNA, 18 bp, Artificial Sequence
  Description of Artificial Sequence Synthetic primer
ctccgaccgtatgcgaca

SEQ ID NO: 144 - DNA, 18 bp, Artificial Sequence
  Description of Artificial Sequence Synthetic primer
aacggtacgcaaccacgt

SEQ ID NO: 145 - DNA, 11 bp, Artificial Sequence
  Description of Artificial Sequence Synthetic oligonucleotide
gcctcttacac

SEQ ID NO: 146 - DNA, 52 bp, Artificial Sequence
  Description of Artificial Sequence Synthetic oligonucleotide
ctgccccgggttcctcattctctnnnnnnnnnnctgctgtacggccaaggcg

SEQ ID NO: 147 - DNA, 10 bp, Artificial Sequence
  Description of Artificial Sequence Synthetic oligonucleotide
atcaagcctc

SEQ ID NO: 148 - DNA, 12 bp, Artificial Sequence
  Description of Artificial Sequence Synthetic oligonucleotide
ctgctgcatgnn

SEQ ID NO: 149 - DNA, 39 bp, Artificial Sequence
  Description of Artificial Sequence Synthetic oligonucleotide
tcgtgtgcttccgaagactatcctgctaagtgttttcac